(19) United States
(12) Patent Application Publication
(10) Pub. No.: US 2014/0128357 A1
(43) Pub. Date: May 8, 2014

(54) METHODS OF PRODUCING AND USING BRASSINOSTEROIDS TO PROMOTE GROWTH, REPAIR AND MAINTENANCE OF SKELETAL MUSCLE AND SKIN

(71) Applicants: Ilya Raskin, Manalapan, NJ (US); Debora Esposito, Concord, NC (US); Slavko Komarnytsky, Concord, NC (US); Thirumurugan Rathinasabapathy, Highland Park, NJ (US); Leonel Rojo Castillo, Iquique (CL)

(72) Inventors: Ilya Raskin, Manalapan, NJ (US); Debora Esposito, Concord, NC (US); Slavko Komarnytsky, Concord, NC (US); Thirumurugan Rathinasabapathy, Highland Park, NJ (US); Leonel Rojo Castillo, Iquique (CL)

(73) Assignee: Rutgers University, New Brunswick, NJ (US)

(21) Appl. No.: 13/993,279

(22) PCT Filed: Dec. 13, 2011
(86) PCT No.: PCT/US11/64663

§ 371 Date: Jan. 17, 2014

Related U.S. Application Data
(60) Provisional application No. 61/422,548, filed on Dec. 13, 2010.

(51) Int. Cl. C07J 9/00; C07J 63/00; A61Q 19/00; A61K 8/63

(52) U.S. Cl. CPC C07J 9/00; A61K 8/63; C07J 63/004; A61Q 19/00
USPC 514/181; 514/450; 514/177; 514/212.04

(57) ABSTRACT

The disclosure relates to methods of using brassinosteroid compounds, including those defined by formula I or a derivative thereof, for inducing an anabolically favorable state for growth, repair, and maintenance of skeletal muscle and skin.

### FIELD

The disclosure generally relates to compounds for inducing an anabolically favored state for muscle and skin and methods for producing and using such compounds for growth, repair, and maintenance of skeletal muscles and skin. More specifically, the compounds comprise brassinosteroids.

### BACKGROUND

Brassinosteroids are plant-specific polyhydroxylated derivatives of 5α-cholestane, structurally similar to cholesterol-derived animal steroid hormones and ecdysteroids from insects. Brassinosteroids are found at low levels in pollen, seeds, leaves, and young vegetative tissues throughout the plant kingdom (Bajguz et al., Phytochemistry 62:1027-1046, 2003). The first biologically active plant brassinosteroid was isolated from the pollen of rapeseed Brassica napus in 1979. The natural occurrence of more than 50 compounds of this group has been reported following the initial discovery (Fujioka et al., Annu. Rev. Plant Biol. 54:137-164, 2003). Brassinosteroids function in cell elongation and cell division, and have been particularly studied in relation to processes such as germination and plant photomorphogenesis.

Similar to animal steroid hormones, brassinosteroids regulate the expression of specific plant genes and complex physiological responses involved in growth, partly via interactions with other hormones, setting the framework for brassinosteroid responses. While animal steroid hormones are perceived by a nuclear receptor family of transcription factors, brassinosteroids signal through a cell surface receptor kinase-mediated signal transduction pathway that includes inactivation of a glycogen synthase kinase 3 (GSK-3)-like kinase, brassinosteroid-insensitive locus 2 (BIN2), by dephosphorylation at a conserved phospho-tyrosine residue pTyr 200. The inactivation of BIN2 allows for accumulation of transcriptional factors brassinazole-resistant 1 (BZR1) and BRI1-EMS-Suppressor 1 (BES1) in the nucleus (Kim et al., Nat. Cell. Biol. 11:1254-126, 2009).

Alpha serine/threonine-protein kinase (AKT) is a serine/threonine kinase that signals downstream of growth factor receptors and phosphoinositide-3 kinase (PI3K). Therefore, growth factor receptors, nutrients, and even muscle contraction increase AKT activity. AKT stimulates glucose uptake, glycogen synthesis, and protein synthesis via AKT/mTOR and AKT/GSK-3β signaling networks, and inhibits apoptosis and protein degradation in skeletal muscle by inactivating FoxO transcription factors. AKT is therefore situated at a critical juncture in muscle signaling where it responds to diverse anabolic and catabolic stimuli.

Very little is known about the effects of brassinosteroids in animals. A natural brassinosteroid and its synthetic derivatives were found to inhibit herpes simplex virus type 1 (HSV-1) and arenavirus, measles, Junin, and vesicular stomatitis virus replication in cell culture. A synthetic brassinosteroid analog prevented HSV-1 multiplication and viral spreading in a human conjunctival cell line with no cytotoxicity and reduced the incidence of herpetic stromal keratitis in mice when administered topically, possibly by the modulation of the response of epithelial and immune cells to HSV-1 infection (Michelini et al., J. Steroid Biochem. Mol. Biol. 108:164-170, 2008). Natural brassinosteroids also inhibited growth of several human cancer cell lines without affecting the growth of normal cells (Malikova et al. Phytochemistry 69:418-426, 2008). 24-Epibrassinolide, the most widely used brassinosteroid in agriculture, has a favorable safety profile. The median lethal dose (LD50) of this compound is higher than 1000 mg/kg in mice and higher than 2000 mg/kg in rats when applied orally or subcutaneously.

28-Homobrassinolide (HB) or (22S,23S)-homobrassinolide (HB) (see FIG. 1) is almost as active as 24-epibrassinolide in inducing plant growth in various bioassay systems. HB is a steroidal lactone initially isolated from pollen of Chinese cabbage Brassica campestris var pekinensis and anthers of Japanese cedar Cryptomeria japonica. It is readily available through chemical synthesis, as its concentration in plants is very low. Plant growth promoting effect of HB is associated with the increased synthesis of nucleic acids and proteins (Bajguz, Plant Physiol. Biochem. 38:209-215, 2000; Kartal et al., Plant Growth Regulation 58:261-267, 2009). In addition, HB activated total protein synthesis, induced de novo polypeptide synthesis, and increased thermotolerance of total protein synthesis in plants subjected to heat shock (Kulaeva et al., “Effect of brassinosteroids on protein synthesis and plant-cell ultrastructure under stress conditions.” In: Brassinosteroids. Washington, D.C.: American Chemical Society; 141-155, 2009).

Anabolic steroids, officially known as anabolic-androgen steroids (AAS) or colloquially simply as “steroids”, are drugs which mimic the effects of the male sex hormones testosterone and dihydrotestosterone. They increase protein synthesis within cells, which results in the buildup of cellular tissue (anabolism), especially in muscles. In short, anabolism results in growth and differentiation of cells and tissues in the body, which result in an increase in muscle mass in the resulting increase in lean body mass. However, there are health risks associated with long-term use or excessive doses of anabolic steroids. These effects include harmful changes in cholesterol levels (increased low-density lipoprotein and decreased high-density lipoprotein), acne, high blood pressure, liver damage (mainly with oral steroids), and dangerous changes in the structure of the left ventricle of the heart.

Thus, there is a need in the art for compounds that have an anabolic effect on the body without causing health risks and negative side effects on the body as described herein above. The following disclosure describes the specifics of such brassinosteroid compounds and methods of using them.

### SUMMARY

The disclosure addresses one or more needs in the art relating to the use of brassinosteroids for selective anabolic effects and improved physical fitness and appearance in healthy animal subjects without detrimental androgenic effects. More specifically, the disclosure relates to methods of using brassinosteroid compounds, including those expressed in formula I, for inducing an anabolically favored state for growth, repair, and maintenance of skeletal muscles and skin in animals, such as mammals, e.g. humans.

The disclosure includes methods for increasing a whole-body anabolic effect in a subject comprising the step of administering to the subject a therapeutically effective amount of a composition comprising a compound of formula I or a derivative thereof:

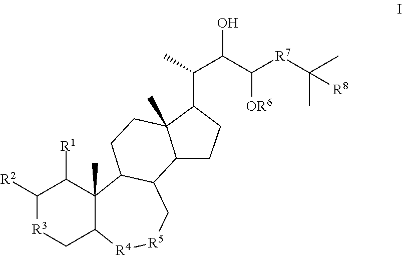

wherein:
R¹ and R² are each independently selected from the group consisting of H and OH;
R³ is selected from the group consisting of C(H)OH, C(H)F, C═O, and C(H)OR⁹;
or R² and R³ together with the carbon atom to which they are bonded form a 3-membered epoxide ring;
R⁴ is selected from the group consisting of CH₂, C═O, C(H)OH, and NH;
R⁵ is selected from the group consisting of a bond, O, NH, and C═O;
R⁶ is selected from the group consisting of H and

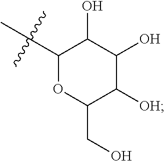 [SMILES: *C1OC(CO)C(O)C(O)C1O]

R⁷ is selected from the group consisting of CH₂, C(H)CH₃, C(H)CH₂CH₃, C═CH₂, and C═C(H)CH₃;
R⁸ is selected from the group consisting of H and CH₃;
R⁹ is selected from the group consisting of C(═O)(CH₂)ₙCH₃ and

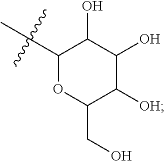 [SMILES: *C1OC(CO)C(O)C(O)C1O]

and
n is selected from the group consisting of 0, 1, 2, 3, 4, 5, 6, 7, 8, 9, 10, 11, 12, 13, 14, 15, 16, 17, and 18.

In some embodiments, the disclosure includes methods for increasing a whole-body anabolic effect in a subject comprising the step of administering to the subject a therapeutically effective amount of a composition comprising a brassinosteroid compound or a derivative thereof. In particular aspects, the brassinosteroid compound is selected from the group consisting of: (22S,23S)-homobrassinolide (HB), (22S,23S)-homocastasterone, (22S,23S)-3α-fluoro-homobrasinolide, (22S,23S)-3α-fluoro-homocastasterone, (22S,23S)-6-aza-homobrassinolide, (22S,23S)-7-aza-homobrassinolide, (22R,23R)-homobrassinolide, (22S,23S)-epibrassinolide, and (22R,23R)-epibrassinolide.

In some aspects, the brassinosteroid compound is (22S,23S)-homobrassinolide (HB), (22S,23S)-homocastasterone, (22S,23S)-3α-fluoro-homocastasterone, (22S,23S)-7-aza-homobrassinolide. In more particular aspects the brassinosteroid compound is (22S,23S)-homobrassinolide (HB),

In some aspects, the whole-body anabolic effect comprises minimal or no androgenic side effect. In some aspects, at least one of the whole-body anabolic effects is an anabolically favorable state for muscle or skin.

In particular aspects, the anabolically favorable state for muscle is measured by increased protein synthesis, increased protein accumulation, or decreased protein degradation in muscle cells. In other aspects, the anabolically favorable state for muscle is measured by increased skeletal muscle mass. In further aspects, the increased skeletal muscle mass is measured by an increased total number of muscle fibers and/or by an increased cross-sectional area of muscle fibers. In even more particular aspects, the increased muscle mass is measured by increased type I and/or type II muscle fibers. In certain aspects, the anabolically favorable state for muscle is measured by increased lean body mass, increased body weight gain, or decreased fat mass. In other aspects, the anabolically favorable state for muscle is measured by increased physical performance, increased physical strength, or increased physical fitness. In another aspect, the increased physical strength is measured by increased grip strength. In a particular aspect, the anabolically favorable state for muscle is measured by increased phosphoryation of AKT.

In other particular aspects, the anabolically favorable state for skin is measured by increased protein synthesis, increased protein accumulation, decreased protein degradation in skin cells, or decreased wound healing time (i.e. wounds heal more quickly). In certain aspects, the increased protein synthesis is measured by increased collagen production. In other aspects, the increased protein accumulation or the decreased protein degradation is measured by increased collagen concentration. In one aspect, the increased protein synthesis is measured by increased elastin production. In another aspect, the increased protein accumulation or the decreased protein degradation is measured by increased elastin concentration. In some aspects, wound healing time was decreased up to 2-fold. In some aspects, the decreased wound healing time is measured by time at which 50% of a cutaneous wound is closed. In some aspects, the decreased wound healing time is measured by percent of original wound size. In some aspects, the decreased wound healing time results from decreased inflammation. In some aspects, the decreased inflammation results from decreased expression of TGF-β messenger RNA, decreased TNF-α messenger RNA, or decreased ICAM-1 messenger RNA. In another aspect, the anabolically favorable state for skin is demonstrated by skin that has increased elasticity, increased smoothness, reduced wrinkles, and/or improved color attributable to healthy infusion of blood.

In further aspects of the disclosure, the compound of formula I, or a particular brassinosteroid compound, is administered in cell culture at a concentration from about 0.01 μM to about 100 μM. In some aspects, the brassinosteroid compound is administered at a concentration from about 0.10 μM to about 30 μM. In more particular aspects, the brassinosteroid compound is administered at a concentration from about 0.30 μM to about 20 μM. In even further aspects, the compound is administered daily.

In other aspects, the compound of formula I, or a particular brassinosteroid compound, is administered at least weekly to the subject at a dosage from about 0.1 mg/kg to about 1000 mg/kg. In some aspects, the compound is administered daily to the subject at a dosage from about 0.1 mg/kg to about 1000 mg/kg. In further aspects, the compound is administered twice daily at a dosage from about 0.1 mg/kg to about 1000 mg/kg. In various aspects, the compound is administered over a period of time, i.e. daily for several weeks or months. In some aspects, the period of time is from days to weeks. In some aspects, the period of time is from days to months.

In other aspects of the disclosure, the brassinosteroid compound is administered topically, parenterally, or enterally. Various means of administering topically, parenterally, or enterally are well known in the art and are described in more detail herein. In a particular aspect, the brassinosteroid compound is topically administered to the skin for cosmetic use. In more particular aspects, the administration is to a mammalian subject. In even more particular aspects, the subject is a human subject.

The disclosure includes various uses of brassinosteroids for increasing a whole-body anabolic effect in a subject according to the disclosure. In some aspects, this anabolic effect is carried out with minimal or no androgenic side effect. In other aspects, the disclosure includes uses of brassinosteroids for the preparation of medicaments for increasing a whole-body anabolic effect in a subject with minimal or no androgenic side effect. Other related aspects are also provided in the instant disclosure.

The foregoing summary is not intended to define every aspect of the subject matter of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the subject matter of the disclosure will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1. Chemical structure of (22S,23S)-homobrassinolide (HB), also known in the art as 28-homobrassinolide (HB).

FIG. 2. Concentration- and time-dependent effects of HB on protein synthesis and degradation in L6 rat myotubes. (A) Cells were incubated for 4 h with [³H]-phenylalanine and treated in triplicate with vehicle (0.1% ethanol), increasing concentrations of HB, or 6.5 nM of IGF-1 as a positive control and protein synthesis was measured as incorporation of [³H]-phenylalanine into protein normalized by total protein. (B) To measure time-dependent effect of HB treatment on protein synthesis, cells were treated with 3 μM HB or 6.5 nM IGF-1 for 1-24 h. (C) Dose-dependent effect of HB on protein degradation was observed in cells labeled overnight with [³H]-phenylalanine and subsequently treated for 4 h with increasing concentrations of HB or 10 nM of insulin as a positive control, and then protein degradation was measured as release of acid-soluble [³H]-phenylalanine into media. (D) For HB time course study of protein degradation, fully differentiated myotubes were treated with 3 μM HB for 1-4 h. Results are expressed as the mean±SEM of determinations performed in triplicate (* P<0.05, ** P<0.01, *** P<0.001 when compared with control by one-way ANOVA and Dunnett's post-test).

FIG. 3. HB increases AKT (Ser473) phosphorylation in L6 myotubes. (A) Representative immunoblot of AKT phosphorylation stimulated with increasing doses of HB or 6.5 nM IGF-1 as a positive control. (B) Representative immunoblot of time-dependent AKT phosphorylation in response to 3 μM HB or 15 min exposure to 6.5 nM IGF-1 as a positive control. Cells were treated with the indicated doses of HB and cell lysates were then analyzed by immunoblotting with phospho- and nonphospho-specific antibodies.

FIG. 4. Effect of HB on body weight gain and food intake in rats fed normal (A-B) and high-protein diets (C-D). Animals received 20 (HB20) or 60 (HB60) mg/kg body weight HB daily for 24 d. Food intake (FI) was recorded daily and cumulative food intake was normalized for 350 g body weight. Results are expressed as the mean±SEM (* P<0.05 when compared to vehicle-treated animals by one-way ANOVA and Dunnett's post-test).

FIG. 5. HB has low androgenic activity. (A) Increasing concentrations of HB or methandrostenolone (positive control, IC50=24 nM) were incubated in the presence of specific androgen receptor binding ligand [³H]-mibolerone for 4 h at 4° C. and DPMs of the incubation buffer were measured to quantify displacement of the ligand. (B) Oral or subcutaneous administration of HB to intact or ORX rats did not affect plasma testosterone levels in animals. Sham-operated or ORX animals received either 20 or 60 mg/kg HB daily for 10 d orally, or 0.4 and 4 mg/kg HB daily for 10 d via subcutaneous injection. No plasma testosterone was detected (ND) in ORX animals and ORX animals treated with HB as compared to a positive control, a subcutaneous injection of 0.4 mg/kg testosterone propionate daily for 10 d. Results are expressed as the mean±SEM (* P<0.05 when compared to vehicle-treated animals by one-way ANOVA and Dunnett's post-test).

FIG. 6. HB (1) increases physical fitness of untrained ORX rats (A), (2) increases mass of mixed-fiber gastrocnemius muscle (B), and (3) induces favorable changes in myofiber type distribution and cross-section area (C-G). ORX rats received vehicle or 20 or 60 mg/kg HB daily for 10 d orally. At the end of the study, the grip strength of hindlimbs and forelimbs of the castrated animals were measured using a digital force gauge. The gastrocnemius muscle was excised, weighed, and serial transverse cryosections of the middle section of the muscle of vehicle-treated animals (C), or animals receiving 20 (D) or 60 (E) mg/kg HB, were stained for mATPase activity to analyze myofiber type distribution (F) and cross-section area (G). Results are expressed as the mean±SEM (* P<0.05 when compared to vehicle-treated animals by one-way ANOVA and Dunnett's post-test).

FIG. 7. Chemical structure of homobrassinolide (1) and its analogs (2-9) used in this study. Compounds (2) and (4) are homocastasterone (B ring is a 6-membered ring).

FIG. 8. Dose-dependent effect of brassinosteroids 1 and 2 on protein synthesis (A) and protein degradation (B) in L6 rat myotubes. (A) Cells were incubated for 4 h with [³H]-phenylalanine and treated in triplicate with vehicle (0.1% ethanol), 6.5 nM of IGF-1 as a positive control, or test compound (0.3-30 μM), and protein synthesis was measured as incorporation of [³H]-phenylalanine into protein normalized by total protein. (B) Dose-dependent effect of HB on protein degradation was observed in cells labeled overnight with [³H]-phenylalanine and subsequently treated for 4 h with vehicle (0.1% ethanol), 10 nM of insulin as a positive control, or brassinosteroid analogs (0.3-30 μM); then protein degradation was measured as release of acid-soluble [³H]-phenylalanine into media. Results are expressed as the mean±SEM of determinations performed in triplicate (* P<0.05, ** P<0.01, *** P<0.001 when compared with control by one-way ANOVA and Dunnett's post-test).

FIG. 9. Cell survival curves as measured by MTT assay for brassinosteroids 1-9 against the murine fibroblast cell line NIH-3T3. Cells were incubated with various concentrations of brassinosteroids (0.3-30 μM) for 24 h at 37° C. The mean absorbance of the control cells represented 100% cell proliferation, and the mean absorbance of treated cells was related to control values to determine sensitivity. Error bars represent standard error (n=6) from mean cell proliferation as determined by repeated experiments.

FIG. 10. Effect of HB and its analogs on AKT (Ser473) phosphorylation in L6 myotubes. Representative immunoblots of AKT phosphorylation stimulated with 10 μM brassinosteroids 1-9 for 1 h or 6.5 nM IGF-1 for 10 min (positive control). Cells lysates normalized to contain 50 μg of total soluble protein were analyzed by immunoblotting with phospho- and nonphospho-specific antibodies.

FIG. 11. Pharmacogenomic effect of HB in vivo. (A) RNA was extracted from pooled (n=5) gastrocnemius muscle samples of control and HB-treated animals (60 mg/kg for 24 d) and analyzed using a rat insulin signaling pathway PCR array to measure relative gene expression levels for 84 genes. Central black line indicates fold changes ((2̂(−ΔCt)) of 1, while the hatched lines indicate the 4-fold change in gene expression threshold. (B) Results for Igf2 gene expression and (C) a set of the myogenic transcriptional factors that modulate muscle growth and differentiation were further confirmed by conventional RT-PCR on individual muscle samples (n=5) from control and HD-treated animals. Results are expressed as the mean±SEM of determinations performed in duplicate (* P<0.05 when compared with control by Student's t test).

FIG. 12. Synthesis of some of the Brassinosteroids used herein is described as set out below: (A) I. Synthesis of (22S,23S,24S)-2α,3α, 22,23-tetrahydroxy-24-ethyl-5α-cholestan-6-one; (B) II. Synthesis of (22S,23S,24S)-3α-fluoro-22,23-dihydroxy-24-ethyl-5α-cholestan-6-one; (C) III. Synthesis of (22S,23S, 24S)-3α-fluoro-22,23-dihydroxy-7-oxo-24-ethyl-5α-cholestan-6-one; (D) IV. Synthesis of (22S,23S,24S)-2α,3α,22,23-tetrahydroxy-24-ethyl-B-homo-6-aza-5α-cholestan-6-one; and (E) V. Synthesis of (22S,23S,24S)-2α,3α,22,23-tetrahydroxy-24-ethyl-B-homo-7-aza-5α-cholestan-6-one.

FIG. 13. Time course of brassinosteroid effect on wound healing in a mouse cutaneous injury model. Test substances were administered topically once daily for 10 consecutive days. Wound closure (%) was determined on days 1, 3, 5, 7, and 9, and wound half closure time (CT50) was obtained. HB increased the percent of wound closure and decreased wound closure time in the mouse model.

FIG. 14. Effect of HB treatment on body weight change associated with wounding. A sharp punch over lumbar spine was applied to remove the skin and vehicle, or 10 μg/mouse of either HB or positive control CGS-21680 was administered topically daily for 10 days. Two-factor repeated-measures ANOVA, *P<0.05 (n=9).

FIG. 15. Time course of wound healing in mouse cutaneous injury model. (A) Wound sizes were photographed and measured every 2 days for 10 days. (B) The wound closure (%) relative to day 1 was determined every 2 days, and CT50 was calculated by linear regression. Two-factor repeated-measures ANOVA, *P<0.05 (n=9).

FIG. 16. Effect of HB on cytokine and chemokine mRNA expression in wounds of C57Bl/6J mice. RNA was isolated from wound tissues collected 10 d post-wounding and mRNA levels for proinflammatory cytokines TNF-α, TGF-β and an adhesion chemokine ICAM-1 were measure by qPCR. The target gene expression of the housekeeping gene (actin) was assigned a value of 1. *p<0.05, **p<0.01 significantly different from vehicle controls, one-way ANOVA with Dunnett's post-hoc test.

FIG. 17. Dose-dependent effect of brassinosteroid treatment on scratch wound closure in vitro. 3T3 Swiss fibroblast monolayers were scratched with a sterile pipette tip and vehicle (0.1% ethanol), FBS (1%, positive control), or various concentrations of (A) HB, (B) (22S,23S)-3α-fluoro-homocastasterone (compound 4), or (C) (22S,23S)-7-aza-homobrassinolide (compound 6) were added to a set of 3 wells per dose and incubated for 12 h. The data represent the average of 2 experiments±SE. * P<0.05; ** P<0.01 (n=3) using one-way ANOVA and Dunnett's post-test.

### DETAILED DESCRIPTION

The disclosure provides methods of using brassinosteroid compounds, including those defined in formula I, for inducing an anabolically favored state for growth, repair, and maintenance of skeletal muscle and skin.

Before any embodiments of the subject matter of the disclosure are explained in detail, however, it is to be understood that the disclosure is not limited in its application to the details of construction and the arrangement of components set forth in the following description or illustrated in the figures and examples. The section headings used herein are for organizational purposes only and are not to be construed as limiting the subject matter described. All references cited in this application are expressly incorporated by reference herein.

The disclosure embraces other embodiments and is practiced or carried out in various ways. Also, it is to be understood that the phraseology and terminology used herein is for the purpose of description and should not be regarded as limiting. The terms “including,” “comprising,” or “having” and variations thereof are meant to encompass the items listed thereafter and equivalents thereof as well as additional subject matter.

### DEFINITIONS

Unless otherwise defined, all technical and scientific terms used herein have the same meaning as commonly understood by one of ordinary skill in the art to which this disclosure belongs. The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

The following abbreviations are used throughout.

AKT Alpha serine/threonine-protein kinase
ANOVA Analysis of variance

### BR Brassinosteroid

### CS Castasterone

CT50 Time at which 50% of the cutaneous wound is closed

### DMEM Dulbecco's Modified Eagle's Medium

DMSO Dimethyl sulfoxide

### EBL 24-Epibrassinolide

### ECL Electrochemiluminescence

EDTA Ethylenediaminetetraacetic acid
ELISA Enzyme-linked immunosorbent assay
FBS Fetal bovine serum

### HB (22S,23S)-homobrassinolide

ICAM Intercellular adhesion molecule
IGF-1 Insulin-like growth factor 1

### ORX Orchiectomized

PBS Phosphate-buffered saline
PCR Polymerase chain reaction
PDGF Platelet-derived growth factor

### PMSF Phenylmethanesulfonylfluoride

### RIPA Radio-Immunoprecipitation Assay Buffer

RNA Ribonucleic acid
SDS Sodium dodecyl sulfate
TGF-β Transforming growth factor-beta
TNF-α Tumor necrosis factor-alpha

It is noted here that, as used in this specification and the appended claims, the singular forms “a,” “an,” and “the” include plural reference unless the context clearly dictates otherwise.

Ranges, in various aspects, are expressed herein as from “about” or “approximately” one particular value and/or to “about” or “approximately” another particular value. When values are expressed as approximations, by use of the antecedent “about,” it will be understood that some amount of variation is included in the range.

As used herein, the following terms have the meanings ascribed to them unless specified otherwise.

The term “anabolic” is understood to represent metabolic processes where complex molecules are synthesized from simpler ones, such as, for example, the synthesis of muscle proteins or skin proteins from amino acids. An “anabolic state” is defined as a state in which nitrogen is differentially retained in lean body mass, either through stimulation of protein synthesis and/or decreased breakdown of protein anywhere in the body (Kuhn, Recent Prog. Horm. Res. 57:411-434, 2002). Anabolic processes tend toward “building up” organs and tissues. These processes produce growth and differentiation of cells and, generally, increases in body size, a process that involves synthesis of complex molecules. Examples of anabolic processes and effects include increases in muscle mass, bone mass, red blood cell production, and increases in synthesis of collagen and elastin in skin.

Additionally, the term “anabolic” includes mechanisms of action which are anti-catabolic processes. The term “catabolic” is understood to represent metabolic processes that are destructive. Such destructive processes involve the breakdown of larger molecules into smaller molecules, such as the breakdown of protein or “protein degradation.” In various aspects of the disclosure, brassinosteroids have “anti-catabolic effects” on protein degradation, i.e. inhibit protein degradation.

The term “whole-body anabolic effect” is understood to represent an overall positive effect on the whole body of a subject. Such an anabolic effect is exhibited by an increase in protein synthesis within cells, which results in the buildup of cellular tissue (anabolism), especially in cells and tissues of muscles and of skin, as well as general effects, such as an increase in strength, endurance, and lean body mass.

The term “anabolically favorable state” refers to a positive state of “building up” wherein protein synthesis is increased, and/or protein degradation is decreased, with an increase in protein accumulation. In some aspects, an anabolically favorable state for muscle is understood to mean that muscle protein synthesis is stimulated and muscle protein accumulates, resulting in muscle that increases in strength and/or size and undergoes shorter recovery periods. In other aspects, an anabolically favorable state for skin is understood to mean that skin protein synthesis is stimulated and skin protein accumulates, resulting in skin that is firmer or has increased elasticity and/or youthful appearance (increased smoothness, reduced wrinkles, and color attributable to healthy infusion of blood). In particular aspects, the anabolically favorable state for skin is understood to mean that the skin appears younger and has less wrinkles. In another particular aspect, the anabolically favorable state for skin is understood to mean that wound healing time is decreased. Consequently, skin heals faster. As a result, because treatment with the compounds of formula I and the brassinosteroid compounds described herein induce an “anabolically favorable state,” the compounds are useful in growth, repair, and maintenance of skeletal muscles and skin.

The term “with minimal or no androgenic side effect” is understood to indicate that there is little or no secondary effect that can be attributed to a male sex steroid, such as factors that are attributable to inherent maleness, including development of male sex organs and a typical male body-hair pattern, as well as undesirable effects, such as male-pattern baldness, prostate enlargement and acne.

The terms “effective amount” and “therapeutically effective amount” each refer to the amount of brassinosteroid compound used to support a whole-body anabolic effect or an anabolically favorable state for muscle or skin in a subject as set forth herein. For example, a therapeutically effective amount, in some aspects of the disclosure, would be the amount necessary to increase muscle mass, increase lean body mass, decrease fat mass, increase physical performance, increase physical strength, increase protein production, increase protein accumulation, decrease protein degradation, or combinations thereof, in the subject

A “control,” as used herein, can refer to an active, positive, negative or vehicle control. As will be understood by those of skill in the art, controls are used to establish the relevance of experimental results, and provide a comparison for the condition being tested.

“AKT” is a serine/threonine protein kinase that plays a key role in multiple cellular processes such as glucose metabolism, cell proliferation, apoptosis, transcription and cell migration. AKT is the key intermediate in the IGF-1 signaling pathway that modulates downstream targets known to regulate protein synthesis and degradation. Activation of a constitutively active AKT in skeletal muscle leads to rapid muscle hypertrophy accompanied by improved metabolism (Izumiya et al., Cell. Metab. 7:159-172, 2008). Because AKT modulates intracellular targets known to regulate protein synthesis and degradation, AKT phosphorylation is measured to determine brassinosteroid effect on AKT activation.

The terms “polypeptide,” “peptide” and “protein” are used interchangeably herein to refer to a polymer of amino acid residues linked via peptide bonds. The term “protein” typically refers to large polypeptides. The term “peptide” typically refers to short polypeptides.

The term “cosmetic use” is understood to mean for use in enhancing the appearance of the body, or some part thereof, e.g. skin. Cosmetics include skin-care creams, lotions, powders, perfumes, lipsticks, nail polish, eye and facial makeup, towelettes, permanent waves, colored contact lenses, hair colors, hair sprays and gels, deodorants, hand sanitizer, baby products, bath oils, bubble baths, bath salts, butters and many other types of products designed to improve the appearance of the body or a body part. In one aspect, the brassinosteroid compounds described herein are formulated into a skin-care cream, lotion, or makeup to improve the appearance of the skin.

The term “over a period of time” is understood to mean for at least several days. In some aspects, the “period of time” can include treatment for several weeks, several months, or even several years as determined to be necessary by a physician.

As used herein, the term “brassinosteroid” or “BR” is understood to represent a class of polyhydrosteroids that have been recognized as a sixth class of plant hormones that includes all natural and synthetic BRs known in the art, including analogs, variants, and derivatives thereof. An “analog,” “variant” or “derivative” is a compound substantially similar in structure and having the same or similar biological activity, albeit in certain instances to a differing degree, to a naturally occurring molecule. A review of the structure, bioactivity, and applications of brassinosteroids was provided by Zullo et al., (Braz. J. Plant Physiol. 14:143-81, 2002), and is incorporated herein by reference in its entirety. Since their discovery, over 70 BR compounds have been isolated from plants (Bajguz, Plant Physiol. Biochem. 45: 95-107, 2007), and is incorporated herein by reference in its entirety.

In some aspects of the disclosure, a brassinosteroid is understood to include a composition comprising a compound of formula I or a derivative thereof:

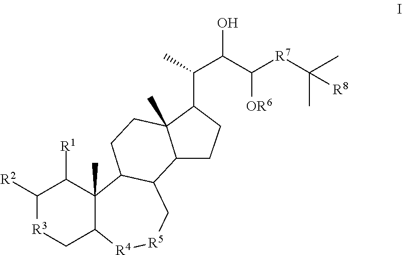

wherein:
R¹ and R² are each independently selected from the group consisting of H and OH;
R³ is selected from the group consisting of C(H)OH, C(H)F, C═O, and C(H)OR⁹;
or R² and R³ together with the carbon atom to which they are bonded form a 3-membered epoxide ring;
R⁴ is selected from the group consisting of CH₂, C═O, C(H)OH, and NH;
R⁵ is selected from the group consisting of a bond, O, NH, and C═O;
R⁶ is selected from the group consisting of H and

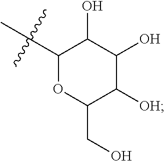 [SMILES: *C1OC(CO)C(O)C(O)C1O]

R⁷ is selected from the group consisting of CH₂, C(H)CH₃, C(H)CH₂CH₃, C═CH₂, and C═C(H)CH₃;
R⁸ is selected from the group consisting of H and CH₃;
R⁹ is selected from the group consisting of C(═O)(CH₂)ₙCH₃ and

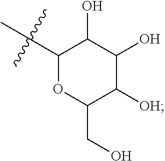 [SMILES: *C1OC(CO)C(O)C(O)C1O]

and
n is selected from the group consisting of 0, 1, 2, 3, 4, 5, 6, 7, 8, 9, 10, 11, 12, 13, 14, 15, 16, 17, and 18.

### Synthesis of Brassinosteroids

In various aspects, BRs are biosynthesized from campesterol. The biosynthetic pathway was elucidated by Japanese researchers and later shown to be correct through the analysis of BR biosynthesis mutants in Arabidopsis thaliana, tomatoes, and peas. The most abundant and widely occurring brassinosteroids are C28 steroids, and among them brassinolide is the most biologically active. Brassinolide was the first isolated brassinosteroid in 1979 when it was shown that pollen from Brassica napus could promote stem elongation and cell division, and the biologically active molecule was isolated.

Plants have multiple pathways for biosynthesis of brassinolide, all derived from the steroid biosynthetic pathway. Two pathways from campestanol to castasterone (CS), C6 oxidation and the late-C6 oxidation pathways, operate in many plants. Another branching pathway, the early-C22 oxidation pathway, was demonstrated using a brassinosteroid-deficient mutant of Arabidopsis thaliana. A shortcut pathway from campesterol to 6-deoxotyphasterol was demonstrated by a functional analysis of cytochrome P450 monooxygenases responsible for brassinosteroid biosynthesis. Thus, at least four pathways are involved in the biosynthesis of CS, and CS is further metabolized to brassinolide (BL) by lactonization of the B ring. Additional explanation is provided by Fujioka et al. (Annu. Rev. Plant Biol. 54:137-64, 2003) and is incorporated herein by reference in its entirety.

In various aspects, the following brassinosteroids are used: [(22S,23S, 24S)-2α,3α,22,23-tetrahydroxy-24 ethyl-β-homo-7-oxo-5α-cholestane-6-one, also known as (22S,23S)-homobrassinolide (HB) or 28-homobrassinolide or “HB”]; (22S,23S,24R)-2α,3α,22,23-tetrahydroxy-24-methyl-B-homo-7-oxa-5α-cholestan-6-one; (22R,23R,24R)-2α,3α,22,23-tetrahydroxy-24-methyl-B-homo-7-oxa-5α-cholestan-6-one; (22S,23S)-homocastasterone; (22S,23S)-3α-fluoro-homobrasinolide; (22S,23S)-3α-fluoro-homocastasterone; (22S,23S)-6-aza-homobrassinolide; (22S,23S)-7-aza-homobrassinolide; (22R,23R)-homobrassinolide; (22S,23S)-epibrassinolide; and (22R,23R)-epibrassinolide.

24-Epibrassinolide (EBL), a brassinosteroid isolated from Aegle marmelos Correa (Rutaceae), in various aspects, is included for use in methods of the disclosure.

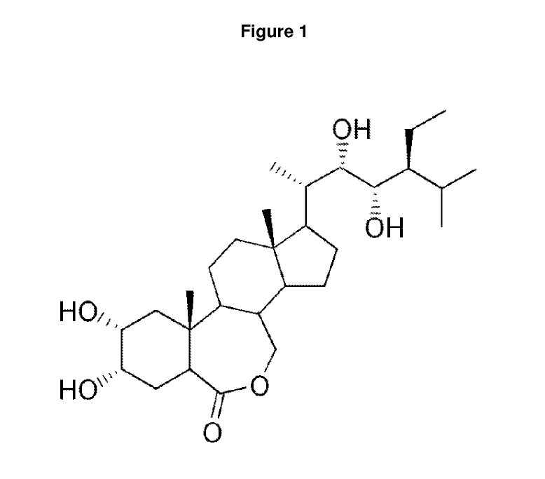

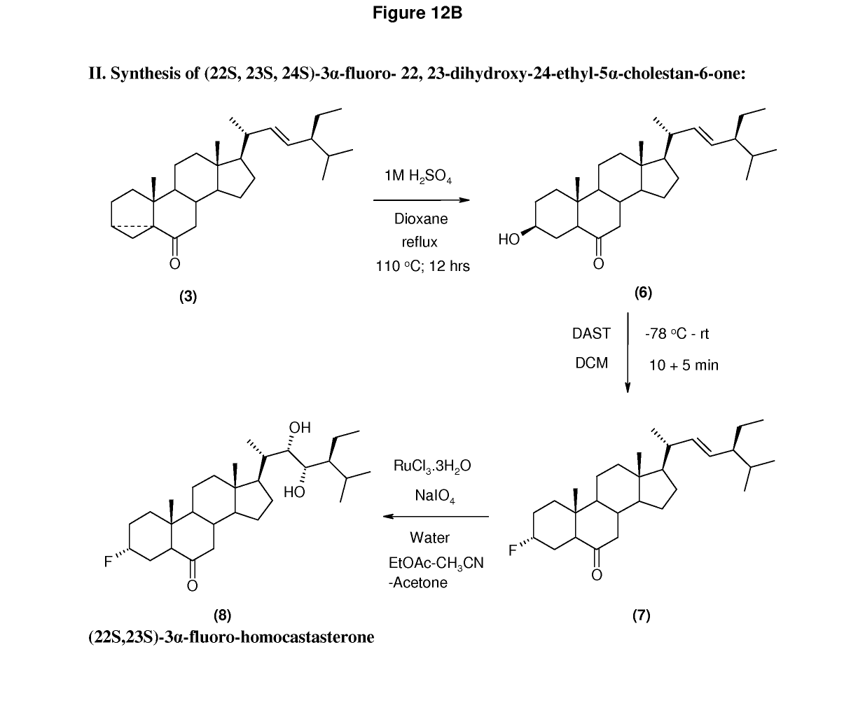

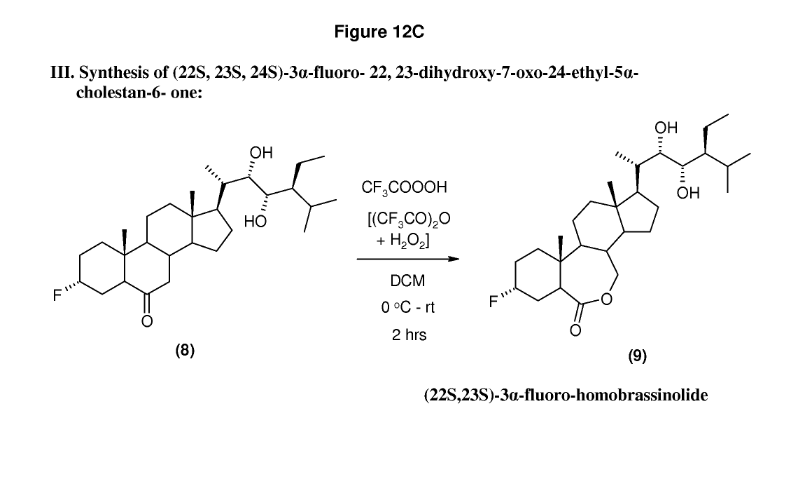

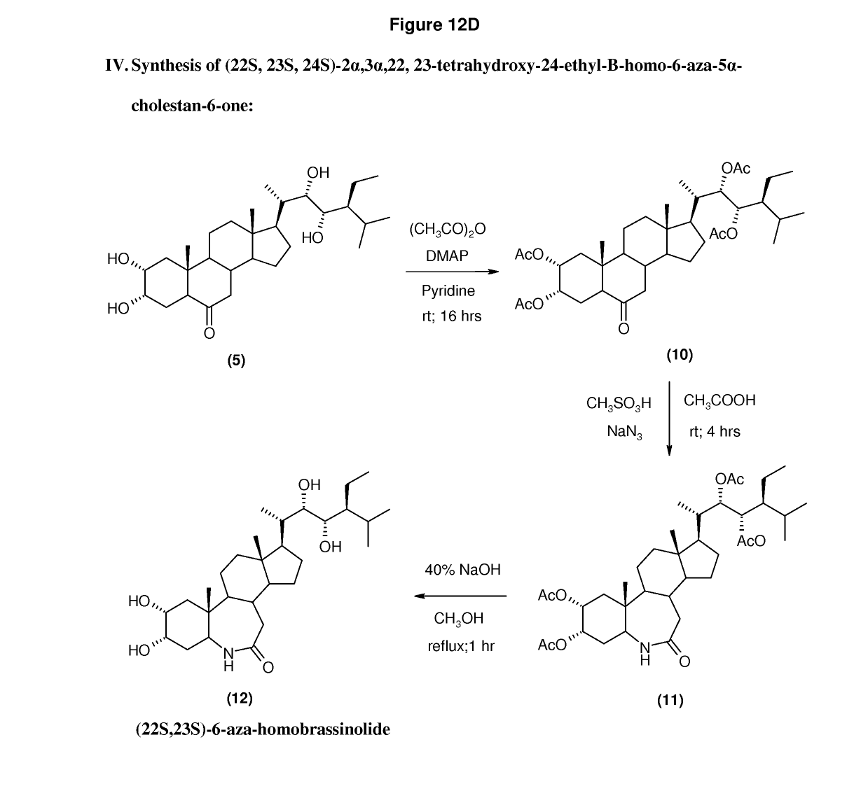

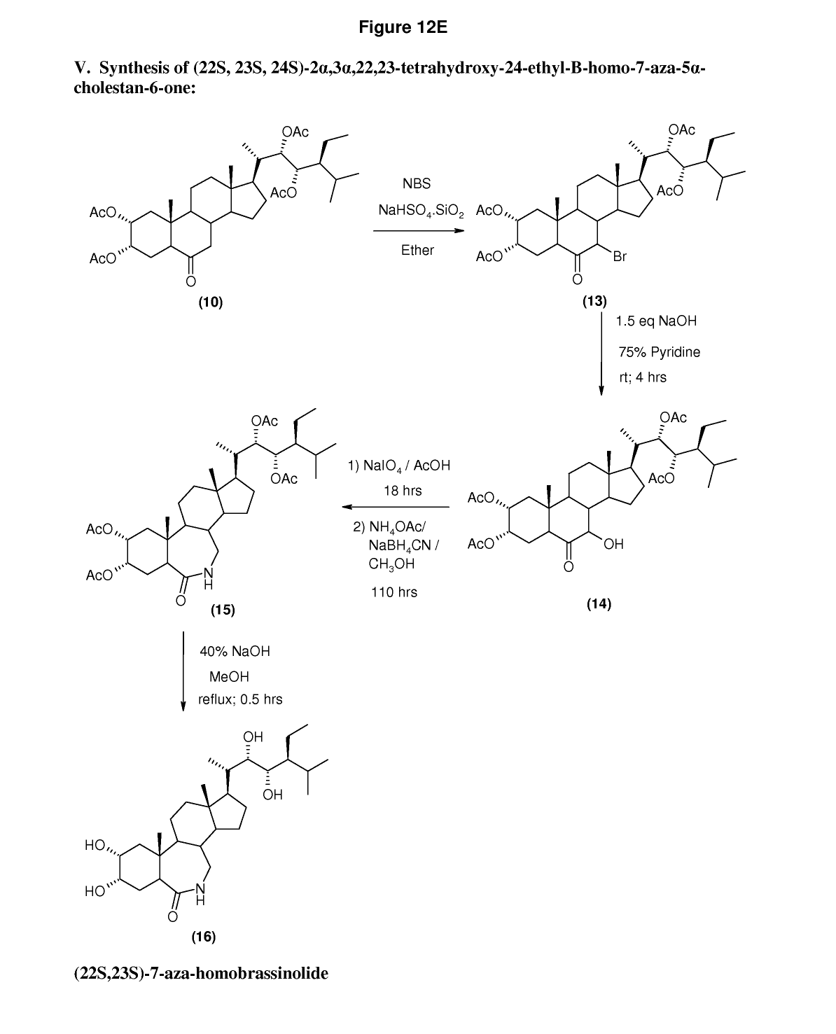

HB (FIG. 1) was purchased from Waterstone Technology (Carmel, Ind.) or SciTech (Praha, Czech Republic) and its structure was confirmed by ESI-LCMS and NMR. (22S,23S,24R)-2α,3α,22,23-tetrahydroxy-24-methyl-B-homo-7-oxa-5α-cholestan-6-one and (22R,23R,24R)-2α,3α,22,23-tetrahydroxy-24-methyl-B-homo-7-oxa-5α-cholestan-6-one were purchased from SciTech (Praha, Czech Republic) and their structures were confirmed by ESI-LCMS and NMR. FIG. 12 describes the synthesis of some of the brassinosteroids used herein.

In some aspects, brassinosteroids are administered to examine their effects on cells. In aspects, the brassinosteroids are administered at concentrations of about 0.01 μM to about 100 μM. In some aspects, the brassinosteroids are administered at concentrations from about 0.1 μM to about 50 μM. In various aspects, the brassinosteroids are administered at concentrations of about 0.2 μM, about 0.3 μM, about 0.4 μM, about 0.5 μM, about 0.6 μM, about 0.7 μM, about 0.8 μM, about 0.9 μM, about 1.0 μM, about 2.0 μM, about 3.0 μM, about 4.0 μM, about 5.0 μM, about 6.0 μM, about 7.0 μM, about 8.0 μM, about 9.0 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, or about 45 μM.

### Chemicals

In various aspects, chemicals were purchased to carry out various experiments relevant to one of more methods described in the disclosure. L-[2,3,4,5,6-3H]-phenylalanine was obtained from GE Healthcare (Piscataway, N.J.). Phospho-AKT and AKT mAbs were purchased from Cell Signaling Technology (Danvers, Mass.). Reagents and enzymes used for qPCR were obtained from Stratagene (La Jolla, Calif.) and Applied Biosystems (Foster City, Calif.). All other chemicals and cell culture media were obtained from Invitrogen (Carlsbad, Calif.) and Sigma (Saint Louis, Mo.) unless specified otherwise.

### Cell Culture

In some aspects, cell culture experiments were carried out. Rat L6 skeletal muscle cell line CRL-1458 was obtained from ATCC (Manassas, Va.). Myoblasts were routinely maintained in Dulbecco's modified Eagle's medium (DMEM) containing 10% FBS and 0.1% penicillin-streptomycin at 37° C. and 5% CO₂. Cells were subcultured into 24-well plates for protein synthesis, degradation, and cell viability studies and into 6-well plates for Western blot analyses (Greiner Bio One, Monroe, N.C.). Once cells reached 90% confluence, differentiation was induced by lowering the serum concentration to 2%, and medium was changed every 2 days. After 7-9 days of culture the myoblasts had fused into multinucleated myotubes (Mandel et al., Nature 251:618-620, 1974). NIH 3T3 murine embryonic fibroblast cell line (ATCC #CCL-92) was maintained in DMEM and 10% FBS at 37° C. in 5% CO₂, and passaged every 3-4 days.

In other aspects, cell culture experiments are carried out to examine the effects of brassinosteroids on skin cells. The culture of human keratinocytes is a convenient and useful model for studies of cellular biology of skin. However, in various aspects, any cell used in the art for the study of skin proteins is used in the disclosure.

In some aspects, the effects of brassinosteroids on collagen and elastin are studied. Collagen and elastin are structural proteins made and used in the human body. Collagen is found primarily in tendons, ligaments, and the connective tissue of skin, blood vessels, and lungs. Elastin is found primarily in the artery walls, lungs, intestines, and skin. These proteins work in partnership in connective tissues. Collagen gives connective tissue and organs rigidity so that they can function, and elastin lets them stretch and return to their original state. Collagen does not allow the elastin to stretch to the point of breaking. In the skin, collagen and elastin are the primary components of the dermis—the layer immediately beneath the epidermis. Collagen and elastin provide the support structure of the skin. Brassinosteroids, in some aspects, have an anabolic effect on such skin proteins, i.e. increase production of collagen and elastin, increase accumulation of collagen and elastin, and decrease breakdown of collagen and elastin.

### Cell Viability Assay and Dose Range Determination

In various aspects, cell viability is measured and the effects of doses of compound and test reagents are tested in cell lines. In some aspects, cell viability was measured by the MTT (3-(4,5-Dimethylthiazol-2-yl)-2,5-diphenyltetrazolium bromide) assay in triplicate (Mosmann et al., J. Immunol. Methods 65:55-63, 1983) and quantified spectrophotometrically at 550 nm using a microplate reader (Molecular Devices, Sunnyvale, Calif.). The concentrations of test reagents that showed no changes in cell viability compared with that of the vehicle (0.1% ethanol) were selected for further studies.

### Measurement of Protein Synthesis

In various aspects, protein synthesis is measured to determine the effect of brassinosteroid on a particular protein. All known methods for measuring protein synthesis are included in the methods of the disclosure. In brassinosteroid dose response studies, fully differentiated myotubes were washed with serum-free DMEM and treated in triplicate with vehicle (0.1% ethanol), increasing concentrations of HB, or 6.5 nM of insulin-like growth factor-1 (IGF-1) as a positive control. Compounds were added to serum-free medium containing 0.5 μCi/mL [³H]-phenylalanine and incubated for 4 h. For the HB time course study, fully differentiated myotubes were treated with 3 μM HB for 1-24 h using the same culture conditions. The incubation was stopped by placing cells on ice, discarding the medium, and washing the cells extensively with ice-cold PBS to remove the non-incorporated radiolabel. Proteins were precipitated with 5% trichloroacetic acid and dissolved in 0.5N NaOH (Montgomery et al., Methods Cell. Sci. 24:123-129, 2002). Specific radioactivity of protein-bound phenylalanine was quantified using liquid scintillation counter LS 6500 (Beckman Coulter, Fullerton, Calif.) and normalized to mg of total protein determined by BCA protein assay (Pierce Biotechnology, Rockford, Ill.).

### Measurement of Protein Degradation

In various aspects, protein degradation is measured to determine the effect of brassinosteroid on a particular protein. All known methods for measuring protein degradation are included in the methods of the disclosure. The effect of brassinosteroids on protein degradation was investigated in fully differentiated myotubes as described by Fawcett et al. (Arch. Biochem. Biophys. 385:357-363, 2001) with slight modifications. Fully differentiated myotubes were incubated for 16 h to allow labeling of cellular proteins with 1.5 μCi/mL [³H]-phenylalanine. Cells were washed twice with PBS to remove the non-incorporated radiolabel and treated for 4 h with vehicle (0.1% ethanol), increasing concentrations of brassinosteroids, or 10 nM insulin in serum-free medium. The incubation was stopped by placing the cells on ice, and protein in the medium was precipitated with 5% trichloroacetic acid. Specific radioactivity of protein-free phenylalanine was quantified using liquid scintillation counter LS 6500 (Beckman Coulter, Fullerton, Calif.) and normalized to mg of total cell protein determined by BCA protein assay (Pierce Biotechnology, Rockford, Ill.).

### Protein Concentration and Protein Detection

In various aspects, methods of protein detection and methods of measuring protein concentration are carried out. All known methods for detecting proteins and measuring protein concentration are included in the methods of the disclosure.

In some aspects, Western blot analyses were carried out. Fully differentiated L6 myotubes were cultured as described above, and whole cell extracts were prepared in ice-cold RIPA buffer supplemented with 10 mM sodium fluoride, 2 mM sodium orthovanadate, 1 mM PMSF, and protease inhibitor cocktail (Sigma) and centrifuged at 12,000 g for 20 min at 4° C. Equal amounts of protein (50 μg) from the supernatants were separated on 10% SDS polyacrylamide gels and blotted onto the nitrocellulose membrane. Western blot detection was carried out with monoclonal phospho-AKT (Ser473) antibodies according to the manufacturer's instructions (Cell Signaling Technology, Danvers, Mass.). After being washed, the blots were incubated with an anti-rabbit peroxidase-labeled secondary antibody and visualized using ECL Western Blotting Detection Reagent (GE Healthcare, Piscataway, N.J.). After being stripped, the same blots were probed with total AKT antibodies to serve as loading controls.

### Androgen Receptor Binding

Rat androgen receptor binding assays were performed by MDS Pharma Services (Taiwan) as described elsewhere (Chang et al., J. Steroid Biochem. 27:123-131, 1987), incorporated herein by reference. Vehicle (1% DMSO), increasing concentrations of HB, or methandrostenolone were incubated in the presence of specific binding ligand [³H]-mibolerone for 4 h at 4° C. and DPMs of the incubation buffer were measured to quantify displacement of the ligand. Each treatment was repeated 2-3 times, and the results were averaged.

### Animal Studies and Gene Expression Studies

Six-week-old male Wistar rats (180-220 g, Charles River Laboratories, MA) were housed in individual chambers in a room maintained at a constant room temperature with 12 h light-dark cycle. Animals had free access to food and water. Animals were allowed to adapt to new conditions for 7 d and handling the animals was carried out daily during this time to reduce the stress of physical manipulation. Animals were randomized into groups according to bodyweight 1 d prior to dosing.

Protocol 1: Three groups of Wistar rats (n=6), fed a normal diet containing 23.9% protein, 10.7% fat, 5.1% fiber, and 58.7% carbohydrates, resulting in 4.61 kcal/g energy value (#5001 Rodent Chow diet, Purina, St. Louis, Mo.), were gavaged daily for 24 d with 1 ml of vehicle (5% DMSO in corn oil), 20 mg/kg or 60 mg/kg body weight of HB. The body weight of each animal and the total amount of food consumed (accounted for spillage) were recorded every 2 d for the duration of the experiment. At the end of the experiment, blood was collected by heart puncture after CO₂ inhalation and animal body composition was assessed by DEXA dual-energy X-ray absorptiometry analysis using a Lunar Prodigy Densitometer (GE Healthcare, Waukesha, Wis.). At necropsy, tissue weights were recorded. Tissue samples were collected by snap-freezing in liquid nitrogen and stored at −80° C. for further studies. Total RNA was isolated using Trizol, its quantity and purity were determined using a NanoDrop (Nanodrop Technologies, Wilmington, Del.). Pooled RNA samples were used for the rat insulin signaling PCR array (Qiagen, Valencia, Calif.) and analyzed according to the manufacturer's protocol. cDNA synthesis and quantitative PCR analysis were performed essentially as described by Komarnytsky et al. (Int. J. Obes. (Lond), 2010 Sep. 7, “Potato protease inhibitors inhibit food intake and increase circulating cholecystokin levels by a trypsin-dependent mechanism,” [Epub ahead of print; PMID: 20820171 [PubMed—as supplied by publisher]].

Protocol 2: Three groups of Wistar rats (n=8), fed a high-protein diet containing 39.4% protein, 10.0% fat, 4.3% fiber, and 37.0% carbohydrates, resulting in 3.93 kcal/g energy value (#5779 diet, Testdiet/Purina, Richmond, Ind.), were gavaged daily for 24 d with 1 ml of vehicle (5% DMSO in corn oil), 20 or 60 mg/kg HB. All procedures and measurements followed protocol 1.

Protocol 3: Four-week-old sham-operated (sham, n=6) or orchiectomized (ORX, n=24) Wistar rats (Charles River Laboratories, MA) were subject to a 10-d Hershberger assay (surgically castrated peri-pubertal adult model) under the following experimental conditions: sham, ORX (vehicle), ORX (20 mg/kg HB orally), ORX (60 mg/kg HB orally), and ORX (0.4 mg/kg testosterone propionate subcutaneously, serving as a positive control for the assay). All procedures and measurements followed protocol 1, except no body composition measurements were taken. Limb grip strength was measured for control animals and animals receiving 60 mg/kg HB using a digital force gauge (Wagner Instruments model FDV5) by Product Safety Laboratories (Dayton, N.J.). After the rats were allowed to grip the screen with paws, the animals were quickly pulled until the paws released from the screen, and the required release force was recorded. Three trials on each animal were performed in triplicate, and significance was determined using Student's t test (p<0.05). In addition to the gastrocnemius muscle, androgen-sensitive tissues (ventral prostate, seminal vesicles, bulbocavernosus/levator ani muscle complex, glans penis, and Cowper's gland) were dissected out and weighed.

Protocol 4: Four-week-old sham-operated (sham, n=6) or orchiectomized (ORX, n=24) Wistar rats (Charles River Laboratories, MA) were subject to a 10-d Hershberger assay (surgically castrated peri-pubertal adult model) under the following experimental conditions: sham, ORX (vehicle), ORX (0.4 mg/kg HB subcutaneously), ORX (4 mg/kg HB subcutaneously), and ORX (0.4 mg/kg testosterone propionate subcutaneously, serving as a positive control for the assay). All procedures and measurements followed protocol 1.

### Skeletal Muscle

Skeletal muscle is a form of striated muscle tissue under control of the somatic nervous system. It is one of three major muscle types, the others being cardiac and smooth muscle. As its name suggests, most skeletal muscle is attached to bones by bundles of collagen fibers known as tendons.

Skeletal muscle is made up of individual components known as muscle fibers. These fibers are formed from the fusion of developmental myoblasts (a type of embryonic progenitor cell that gives rise to a muscle cell). The myofibers (muscle fiber) are long, cylindrical, multinucleated cells composed of actin and myosin myofibrils repeated as a sarcomere, the basic functional unit of the cell that is responsible for skeletal muscle's striated appearance and that forms the basic machinery necessary for muscle contraction. The term “muscle” refers to multiple bundles of muscle fibers held together by connective tissue.

### Muscle Fibers

Individual muscle fibers are formed during development from the fusion of several undifferentiated immature cells known as myoblasts into long, cylindrical, multi-nucleated cells. Differentiation into this state is primarily completed before birth with the cells continuing to grow in size thereafter. Skeletal muscle exhibits a distinctive banding pattern when viewed under the microscope due to the arrangement of cytoskeletal elements in the cytoplasm of the muscle fibers. The principal cytoplasmic proteins are myosin and actin (forming “thick” and “thin” filaments, respectively) which are arranged in a repeating unit called a sarcomere. The interaction of myosin and actin is responsible for muscle contraction.

There are two principal ways to categorize muscle fibers: the type of myosin (fast or slow) present, and the degree of oxidative phosphorylation that the fiber undergoes. Skeletal muscle can thus be broken down into two broad categories: Type I and Type II. Type I fibers appear red due to the presence of the oxygen binding protein myoglobin. Type I fibers are suited for endurance and are slow to fatigue because they use oxidative metabolism to generate ATP. Type II fibers are white due to the absence of myoglobin and a reliance on glycolytic enzymes. Type II fibers are efficient for short bursts of speed and power and use both oxidative metabolism and anaerobic metabolism depending on the particular sub-type, and they are quicker to fatigue.

Individual muscles are a mixture of three types of muscle fibers (type I, type IIa and type IIb), but their proportions vary depending on the action of that muscle. If a weak contraction is needed only the type 1 motor units will be activated. If a stronger contraction is required the type IIa fibers will be activated or used to assist the type I fibers. Maximal contractions facilitate the use of type IIb fibers which are always activated last. These fibers are used during ballistic activities but tire easily.

### Muscle Histology

The muscle samples for histochemical analysis were taken from the middle section of the mixed-fiber gastrocnemius muscle of the castrated animals treated according to protocol 4 (described herein above) to allow for the observation of differences in fiber type distribution and cross-section area associated with ORX and HB treatments. Serial transverse cryosections (10 μm) were prepared from each muscle and were analyzed for myofibrillar adenosine triphosphatase (mATPase) histochemistry after alkaline (pH=9.5) preincubation. Fiber cross-section area and enzyme activity levels were determined from digitized images of the muscle cross-sections that were stored as gray-level pictures using ImageJ software (National Institutes of Health, Bethesda, Md.).

### Assays of Plasma Samples

Blood samples were taken from overnight-fasted animals by heart puncture, collected in EDTA-coated tubes, centrifuged 1,500 g for 20 min, and separated plasma was stored at −80° C. until analysis. Glucose was measured in blood samples using a Lifescan glucometer (Johnson and Johnson, New Brunswick, N.J.). Plasma concentrations of insulin were determined by a rat/mouse insulin ELISA kit (Millipore, Billerica, Mass.). Plasma triglycerides and total cholesterol were measured by enzymatic colorimetric assays (Wako Diagnostics, Richmond, Va.). Total testosterone in plasma samples was quantified by an ELISA assay (DRG Diagnostics, Marburg, Germany).

### Statistics

Statistical analyses were carried out using Prism 4.0 (Graph Pad Software, San Diego, Calif.). Unless otherwise noted, data were analyzed by a one-way ANOVA with treatment as a factor. Post-hoc analyses of differences between individual experimental groups were made using the Dunnett's multiple comparison test. Body weight gain was analyzed by two-factor repeated-measures ANOVA, with time and treatment as independent variables. Significance was set at p<0.05. Values were reported as means±SEMs. The 50% inhibitory concentration (IC50) was calculated by a nonlinear regression curve analysis.

### Routes of Administration and Dosages

The disclosure contemplates compositions comprising a biologically active compound, i.e. brassinosteroid compound (or the brassinosteroid compound alone), that are available for topical, enteral, or parenteral administration. For topical administration, the compound, in various aspects, is delivered transdermally, transmucosally, epicutaneously, via eye drops, via ear drops, or by inhalation. In particular aspects, the compound is applied transdermally to the skin for cosmetic purposes. For enteral/parenteral administration, the compound, in various aspects, is delivered orally, rectally, sublingually, sublabially, buccally, by injection, or by infusion. In particular aspects, when the compound is injected, it is injected intravenously, intraarterially, subcutaneously, intradermally, intramuscularly, intracardiacly, pericardially intrathecally, intraperitoneally, intravesically, intravitreally, intravaginally, epidurally, or intranasally.

In some aspects, the biologically active compound(s) is tabletted, encapsulated or otherwise formulated for oral administration. In some aspects, the compositions are provided as pharmaceutical compositions, nutraceutical compositions (e.g., a dietary supplement), or as a food or beverage additive, as defined by the U.S. Food and Drug Administration. The dosage form for the above compositions are not particularly restricted. For example, liquid solutions, suspensions, emulsions, tablets, pills, capsules, sustained release formulations, powders, suppositories, liposomes, microparticles, microcapsules, sterile isotonic aqueous buffer solutions, and the like are all contemplated as suitable dosage forms.

In some aspects, the compositions include one or more suitable diluents, fillers, salts, disintegrants, binders, lubricants, glidants, wetting agents, controlled release matrices, colorants, flavorings, carriers, excipients, buffers, stabilizers, solubilizers, commercial adjuvants, and/or other additives known in the art.

In various aspects, any pharmaceutically acceptable (i.e., sterile and acceptably non-toxic as known in the art) liquid, semisolid, or solid diluent that serves as a pharmaceutical vehicle, excipient, or medium is used. In particular aspects, diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. In some aspects, such compositions influence the physical state, stability, rate of in vivo release, and rate of in vivo clearance of the compound.

Pharmaceutically acceptable fillers, in certain aspects, include, lactose, microcrystalline cellulose, dicalcium phosphate, tricalcium phosphate, calcium sulfate, dextrose, mannitol, and/or sucrose. In other aspects, salts, including calcium triphosphate, magnesium carbonate, and sodium chloride, are used as fillers in the pharmaceutical compositions.

In some aspects, binders are used to hold the composition together to form a hard tablet. In particular aspects, binders include materials from organic products such as acacia, tragacanth, starch and gelatin. Other suitable binders include methyl cellulose (MC), ethyl cellulose (EC) and carboxymethyl cellulose (CMC).

In some aspects, the compound is administered in a food product. A food product comprising the compound further comprises a bioavailability enhancer, which acts to increase the absorption of the compound by the body. Bioavailability enhancers are natural or synthetic compounds. In one aspect, the food product comprising the compound further comprises one or more bioavailability enhancers in order to enhance the bioavailability of the compound.

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.). In some aspects, a natural bioavailability enhancer is present in an amount of from about 0.02% to about 0.6% by weight based on the total weight of the food product.

Examples of suitable synthetic bioavailability enhancers include, but are not limited to, Gelucire®., Labrafil® and Labrasol®, Lauroglycol®, Pleurol Oleique® (Gattefosse Corp., Paramus, N.J.) and Capmul® (Abitec Corp., Columbus, Ohio).

The amount and administration regimen of the compound is based on various factors relevant to the purpose of administration, for example subject age, sex, body weight, hormone levels, or other nutritional need of the subject.

In some aspects, the compound is administered to a subject at a dosage, i.e. amount, from about 0.001 mg/kg body weight to about 10 g/kg body weight. In some aspects, the compound is administered to the subject in an amount of about 0.005 mg/kg body weight. In some aspects, the compound is administered to the subject in an amount of about 0.01 mg/kg body weight, about 0.02 mg/kg body weight, about 0.03 mg/kg body weight, about 0.04 mg/kg body weight, about 0.05 mg/kg body weight, about 0.06 mg/kg body weight, about 0.07 mg/kg body weight, about 0.08 mg/kg body weight, about 0.09 mg/kg body weight, about 0.1 mg/kg body weight, about 0.2 mg/kg body weight, about 0.3 mg/kg body weight, about 0.4 mg/kg body weight, about 0.5 mg/kg body weight, about 0.6 mg/kg body weight, about 0.7 mg/kg body weight, about 0.8 mg/kg body weight, about 0.9 mg/kg body weight, about 1 mg/kg body weight, about 2 mg/kg body weight, about 3 mg/kg body weight, about 4 mg/kg body weight, about 5 mg/kg body weight, about 6 mg/kg body weight, about 7 mg/kg body weight, about 8 mg/kg body weight, about 9 mg/kg body weight, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, about 100 mg/kg body weight, about 150 mg/kg body weight, about 200 mg/kg body weight, about 250 mg/kg body weight, about 300 mg/kg body weight, about 350 mg/kg body weight, about 400 mg/kg body weight, about 450 mg/kg body weight, about 500 mg/kg body weight, about 550 mg/kg body weight, about 600 mg/kg body weight, about 650 mg/kg body weight, about 700 mg/kg body weight, about 750 mg/kg body weight, about 800 mg/kg body weight, about 850 mg/kg body weight, about 900 mg/kg body weight, about 950 mg/kg body weight, about 1 g/kg per body weight, about 2.5 g/kg body weight, about 5 g/kg body weight, about 7.5 g/kg body weight, or about 10 g/kg body weight.

In some aspects, the compound is administered to animal subjects at a dosage from about 1 μg/kg body weight per day to about 10 g/kg body weight per day. In particular aspects, the compound is administered to animal subjects at a dosage from about 1 mg/kg body weight per day to about 1 g/kg body weight per day. In more particular aspects, the compound is administered to animal subjects at a dosage from about 10 mg/kg body weight per day to about 100 mg/kg body weight per day.

In particular aspects, a regimen comprises multiple doses of the compound. In one aspect, the compound regimen as set out above is administered once per day. In some aspects, the compound is administered daily over a period of several days, several weeks, several months, or several years. In various aspects, the compound is administered to an individual at any time. In some aspects, the compound is administered concurrently, or prior to or after the consumption of a meal.

It will be appreciated that the compound described herein is useful in the fields of human medicine and veterinary medicine to provide a brassinosteroid compound to a subject in need thereof. In some aspects, the subject or individual to be treated is a mammal. In particular aspects, the mammal is a human. For veterinary purposes, subjects include mammals and non-mammals. In certain aspects, the mammals include farm animals, such as cows, sheep, pigs, horses, and goats. In various aspects, the mammals include companion animals, such as dogs and cats. In other aspects, the subjects include exotic and/or zoo animals, which can be mammals and non-mammals. In other aspects, the subjects include laboratory animals, such as mice, rats, rabbits, guinea pigs, and hamsters. In certain aspects, the non-mammals include poultry, such as chickens, turkeys, ducks, and geese.

In one aspect, the compound is formulated for administration to humans and thus contains flavors that would appeal to humans, such as fruit-based flavors. A compound that is formulated with confectionery-like qualities and flavors is also appealing to children who are often resistant to taking medications or supplements due to unpleasant tastes or texture.

In another aspect, the compound is formulated for administration to a non-human animal. Administration of the compound to an animal in conventional solid dosage forms, such as tablets and capsules, can be problematic in that the animal often expels them, and multiple dosing is often difficult because the animal learns to resist the dosing procedure. When formulated for this purpose, the compound, in various aspects, contains flavors that more typically appeal to non-human animals, for example, fish or meat flavors.

Each publication, patent application, patent, and other reference cited herein is incorporated by reference in its entirety to the extent that it is not inconsistent with the present disclosure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Additional aspects and details of the disclosure will be apparent from the following examples, which are intended to be illustrative rather than limiting.

### Example 1

### The Effect of HB on Protein Synthesis

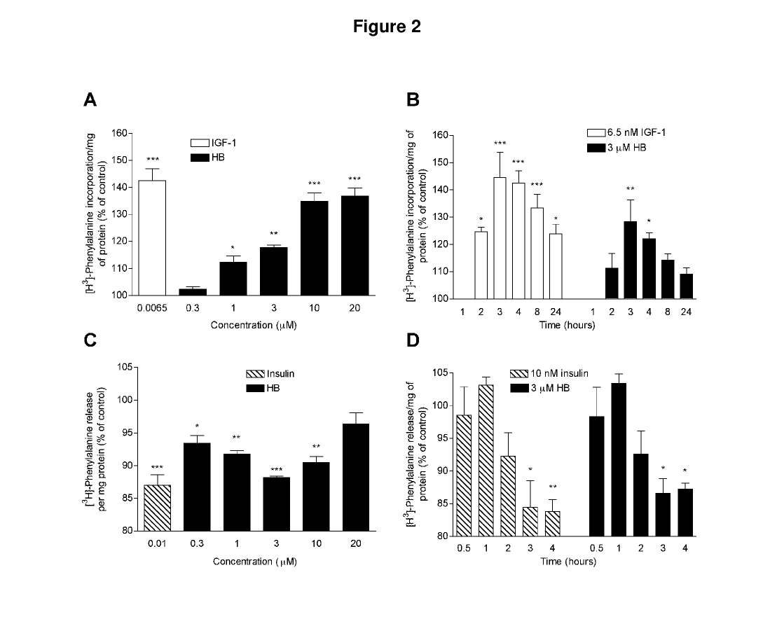

To determine whether HB induces protein synthesis, cells were treated with several concentrations of HB (0.3-20 μM) for 4 h and the incorporation of radiolabelled [³H]-phenylalanine in myotubes was assessed. At a lower concentration (1 μM), HB increased protein synthesis by 12.4±2.3% above control levels (p<0.05). A response approached saturation between 10 and 20 μM of HB, with increases of 34.9±3.1% and 36.9±2.9%, respectively (FIG. 2A). IGF-1 at 6.5 nM served as positive control in this assay. IGF-1 (positive control) increased protein synthesis by 42.5±4.5%. Higher concentrations of HB were less effective. HB showed no toxicity to fully differentiated L6 rat skeletal myotubes up to 25 μM as established by the MTT assay and cytological observations.

To investigate a kinetic effect of HB on protein synthesis, a 1-24 h study was carried out with 3 μM HB, selected as a 50% effective dose to treat myotubes. A time-dependent increase in protein synthesis in response to HB was observed (FIG. 2B). HB-stimulated protein synthesis peaked at 3 h and started to decrease after 4 h of treatment. A similar kinetic effect of IGF-1 on protein synthesis was observed, although the effect of IGF-1 was of a greater magnitude.

This study shows that HB dose- and time-dependently stimulated protein synthesis in myotubes.

### Example 2

### The Effect of 28HB on Protein Degradation

In order to assess whether HB affects protein degradation, the degradation of proteins labeled with [³H]-phenylalanine was monitored for the release of acid-soluble radioactivity into the medium. HB at concentrations of 0.3-20 μM inhibited protein degradation dose-dependently and the effect of HB reached a plateau at a concentration between 3 and 10 μM (FIG. 2C).

At the lower concentration, 1 μM HB decreased protein degradation by 8.2±0.6% above control levels (p<0.05). At the higher concentration, 10 μM HB decreased protein degradation by 9.5±0.9% above control levels (p<0.05). Insulin at 10 nM served as positive control in this assay; it reduced protein degradation by 13.0±1.6%. To investigate the kinetics of protein degradation in response to HB, a 1-4 h study was performed with 3 μM HB. Suppression of protein degradation occurred time-dependently and reached a plateau at 3 h for both HB and insulin (FIG. 2D)

IGF-1 has both an anabolic effect on protein synthesis and an anti-catabolic effect on protein degradation in skeletal muscle, similar to insulin (Harper et al., J. Endocrinol. 112:87-96, 1987). Protein synthesis is more sensitive to IGF-1 infusion than to insulin infusion, and is not mediated by insulin receptors (Douglas et al., J. Clin. Invest. 88:614-622, 1991). On the contrary, insulin affects protein turnover by inhibiting protein degradation (Rooyackers et al., Annu. Rev. Nutr. 17:457-485, 1997).

This study shows that HB inhibited protein degradation in L6 rat skeletal muscle cells.

### Example 3

### HB Stimulates Phosphorylation of AKT

It has been shown previously that IGF-1 inhibits protein degradation in myotubes through PI3K/AKT/GSK-3β and PI3K/AKT/mTOR-dependent mechanisms (Li et al., Int. J. Biochem. Cell. Biol. 37:2207-2216, 2005). AKT has been demonstrated to be a key intermediate in the IGF-1 signaling pathway that modulates downstream targets known to regulate protein synthesis and degradation (Hajduch et al., Diabetes 47:1006-1013, 1998). To characterize the transduction pathway through which HB signals to induce positive net protein balance, the phosphorylation level of AKT in L6 myotubes was investigated.

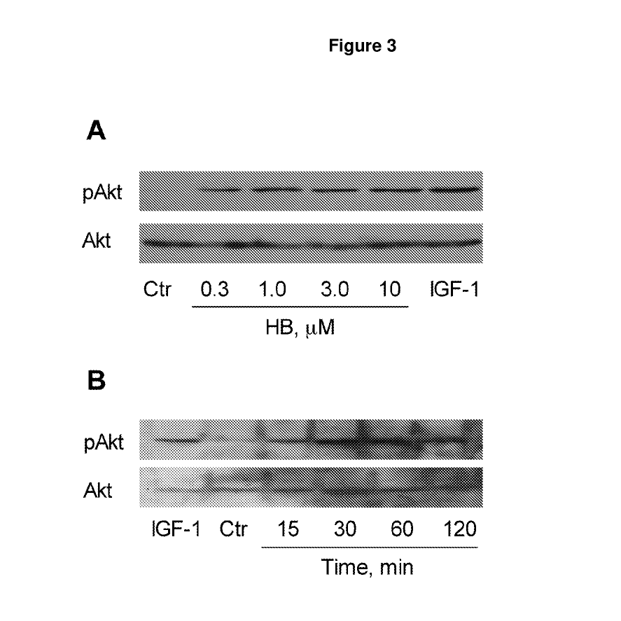

Consistent with the results obtained with the [³H]-phenylalanine incorporation assay, HB stimulated phosphorylation of AKT in a dose- and a time-dependent manner (FIG. 3). Increasing concentrations of HB stimulated Ser473 phosphorylation of AKT up to 3-fold with 3 μM HB after 1 h of treatment (FIG. 3A). AKT stimulation was detected at 30 min after addition of HB, and phosphorylation was maintained up to 1 h, whereas total AKT protein levels were unaltered (FIG. 3B). Although the effect of HB on AKT phosphorylation is not as robust as that described for IGF-1 (Rommel et al., Nat Cell Biol 3:1009-1013, 2001), these data support a role for the PI3K/AKT pathways in HB stimulation of anabolic signaling in L6 myotubes.

This study shows, therefore, that HB inhibited protein degradation in L6 rat skeletal muscle cells, in part by inducing AKT phosphorylation. The effective HB concentrations that produced AKT activation were comparable with the concentrations required to modulate protein synthesis, suggesting that HB involves AKT activation in stimulation of protein synthesis and suppression of protein degradation.

### Example 4

### Anabolic Effects of HB on Body Composition

To evaluate the potential anabolic effects of plant brassinosteroids on body composition of animals, 20 and 60 mg/kg body weight of HB (HB20 and HB60, respectively) were orally administered daily to healthy rats fed normal diet for 24 d.

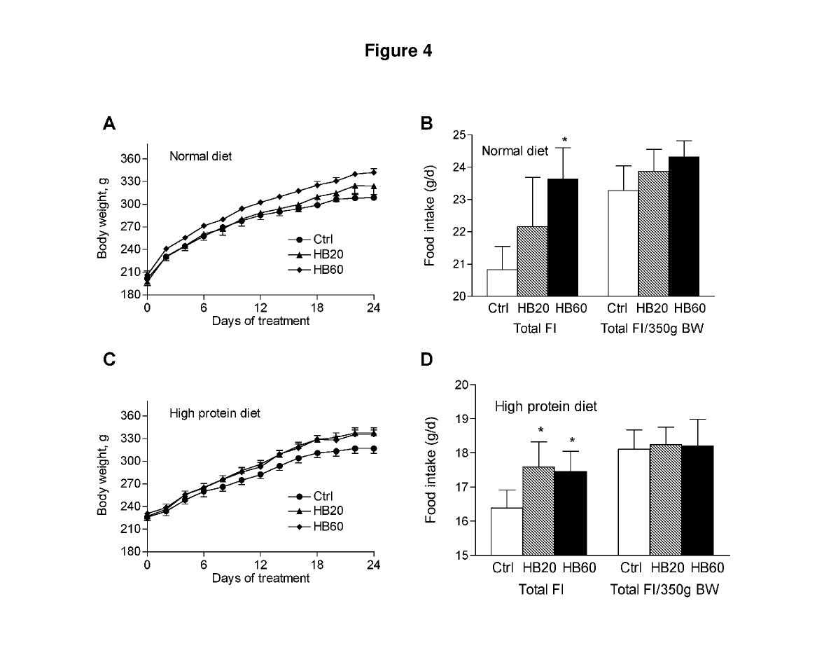

By the end of the treatment, the total body weight gain relative to initial body weight in rats treated with HB20 or HB60 was increased by 18.3% and 26.8%, respectively, compared with vehicle-treated controls (FIG. 4A). A slight but statistically significant increase in total daily food intake (20.8±0.4 g for controls; 22.2±0.8 g for HB20; and 23.6±0.5 g for HB60 group) was associated with HB administration, but when adjusted for body weight, food intake did not differ among all groups (FIG. 4B). Therefore, increases in body weight gain in the HB-treated groups could not be attributed to changes in animal feeding habits. Body composition determined by DEXA analysis showed that increase in lean body mass was significantly greater in HB20 (7.0%) and HB60 animals (14.2%). Fat mass was slightly less in HB20 (−3.9%) and HB60 groups (−4.9%) versus their control counterparts. Thus, the greater body weight gain in the HB-treated rats was predominantly due to increased lean mass (Table 1).

TABLE 1
Body composition and blood biochemistry of rats treated with HB.

| | Normal diet | | | High-protein diet | | |
|---|---|---|---|---|---|---|
| | Control | HB20 | HB60 | Control | HB20 | HB60 |
| Body weight, g | 308.8 ± 4.2 | 324.3 ± 11.5 | 342.3 ± 4.9* | 316.9 ± 6.5 | 337.6 ± 6.4 | 335.8 ± 5.1 |
| Body weight | 107.0 ± 2.9 | 126.6 ± 7.1 | 135.7 ± 9.4* | 76.2 ± 5.7 | 98.3 ± 5.2* | 91.1 ± 3.2* |
| gain, g | | | | | | |
| Lean mass, g | 250.8 ± 9.1 | 268.3 ± 9.0 | 286.5 ± 4.3* | 232.8 ± 6.9 | 246.8 ± 7.4 | 258.6 ± 2.7* |
| Fat mass, g | 51.0 ± 5.9 | 49.0 ± 4.3 | 48.5 ± 3.8 | 62.7 ± 1.6 | 68.6 ± 3.1 | 61.2 ± 3.8 |
| Bone mineral | 7.1 ± 0.2 | 7.0 ± 0.3 | 7.4 ± 0.1 | 7.0 ± 0.1 | 7.5 ± 0.2 | 7.4 ± 0.1 |
| content, g | | | | | | |
| Gastrocnemius | 1.79 ± 0.06 | 2.07 ± 0.06 | 2.13 ± 0.12* | 1.90 ± 0.04 | 1.98 ± 0.01 | 2.17 ± 0.03*** |
| muscle, g | | | | | | |
| Glucose, mM | 5.0 ± 0.2 | 5.1 ± 0.5 | 4.5 ± 0.3 | 5.2 ± 0.1 | 5.1 ± 0.2 | 4.8 ± 0.2 |
| Insulin, ng/ml | 1.92 ± 0.16 | 2.39 ± 0.58 | 2.65 ± 0.45 | 1.80 ± 0.27 | 2.39 ± 0.34 | 2.38 ± 0.41 |
| Cholesterol, mg/dl | 69.0 ± 3.2 | 69.9 ± 6.0 | 84.2 ± 5.3 | 89.2 ± 4.2 | 97.5 ± 6.0 | 87.4 ± 2.9 |
| Triglycerides, mM | 1.9 ± 0.2 | 1.8 ± 0.2 | 1.8 ± 0.5 | 1.4 ± 0.1 | 1.2 ± 0.1 | 1.3 ± 0.1 |

Rats were fed either normal (23.9% protein content) or high-protein (39.4% protein content) diet, and gavaged daily with 20 or 60 mg/kg body weight HB for 24 d. Body composition was measured by DEXA. Results are expressed as the mean ± SEM (*P < 0.05, **P < 0.01, ***P < 0.001 when compared with the appropriate control by one-way ANOVA and Dunnett's post-test).

Administration of HB increased gastrocnemius muscle mass by 15.6% and 19.0% in HB20 and HB60 animals, respectively. Total body bone mineral content (BMC) was slightly greater in HB-treated animals but the difference did not reach significance. Supplementation with HB had no effect on basal plasma cholesterol or triglycerides. Greater doses of HB were associated with slightly lower plasma glucose levels (4.5±0.3 mM) versus controls (5.0±0.3 mM), but the difference did not reach statistical significance. Insulin levels were slightly elevated (Table 1).

### Example 5

### Anabolic Effects of HB in Rats Fed a High-Protein Diet

It has been shown that short-term increases in dietary protein can favor lean body mass and reduce body fat in rats, possibly due to initial decrease in food intake that gradually returns to normal with time (Jean et al., J. Nutr. 131:91-98, 2001). In order to investigate whether high-protein diet can further enhance HB-associated effect on lean mass and muscle mass, rats fed high-protein diet (39.4% protein) were orally administered 20 and 60 mg/kg body weight HB daily for 24 d.

Control animals fed high-protein diet consumed less food and gained less weight than control animals on normal diet (Table 1). Stimulatory effects of HB on body weight and food consumption were apparent on the background of both normal and high-protein diet. The high-protein diet possibly enhanced the stimulatory effect of the lower dose of HB (20 mg/kg) on body weight gain (FIG. 4C). No HB-associated increase in food intake was observed in these animals (FIG. 4D). There were no additional differences in body composition or blood biochemistry that could be attributed to high-protein diet (Table 1).

Oral 24-d administration of HB to healthy rats selectively increased body weight gain, lean body mass, and gastrocnemius muscle mass as compared to vehicle-treated controls (FIG. 4 and Table 1). Supplementation of HB-treated animals with high-protein diet enhanced the effect of the lower dose of HB (Table 1). As expected (Jean et al., supra, 2001), control animals fed high-protein diet (FIGS. 4C and D) exhibited decreased body weight gain, food intake, and other body composition parameters compared to control animals fed normal diet (FIGS. 4A and B). Their plasma triglycerides were also decreased (Table 1). Treatment with HB did not modify blood biochemistry in animals fed either normal or high-protein diet, with the exception of fasting glucose that was slightly lower in cohorts receiving higher doses of HB.

### Example 6

### HB does not Bind Androgen Receptor

Since HB produced anabolic effects similar to those of androgens, a study was carried out to rule out the possibility that HB activates androgen receptor. A binding assay measuring the displacement of the labeled [³H]-mibolerone from the rat nuclear androgen receptor was used to compare HB with methandrostenolone, an androgen analog used therapeutically as an anabolic agent (Feldkoren et al., J. Steroid Biochem. Mol. Biol. 94:481-487, 2005).

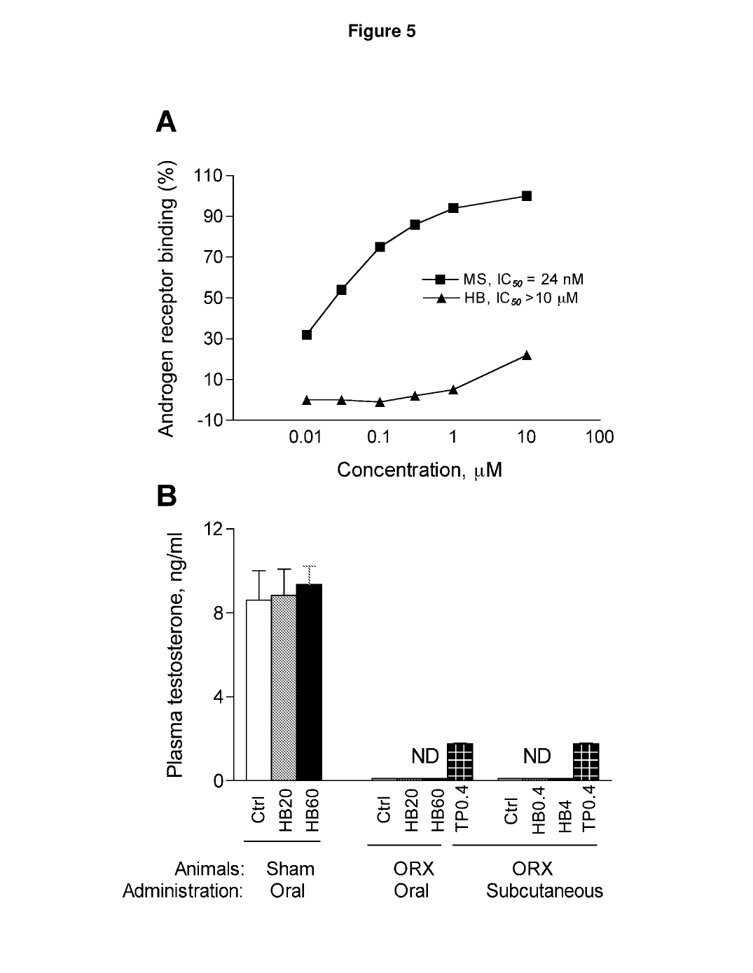

Methandrostenolone produced specific binding to the androgen receptor with an IC50 of 24 nM, and a binding curve similar to the endogenous ligand, testosterone. However, HB showed no significant binding from concentrations of 0.01 μM up to 10 μM (FIG. 5A). This study showed that the in vivo action of HB on body composition and bone could not be attributed to endogenous testosterone action, as plasma testosterone levels did not differ in response to HB treatment (FIG. 5B).

### Example 7

### Selective Effects of HB in ORX Rats

All steroids that are anabolic are derivatives of testosterone and are androgenic as well as anabolic, as they stimulate growth and function of the male reproductive system. Individual drugs vary in their balance of anabolic/androgenic activity but none of the currently available drugs are purely anabolic (Kuhn, Recent Prog. Horm. Res. 57:411-434, 2002). Therefore, the ability of HB versus injected testosterone propionate (positive control) to restore androgen-dependent tissues after androgen deprivation was investigated in a surgically castrated peri-pubertal rat model (Hershberger et al., Proc. Soc. Exp. Biol. Med. 83:175-180, 1953).

Oral and subcutaneous treatments at appropriate dose ranges were initiated 2 wk after orchiectomy (ORX) and continued for 10 d. As expected, androgen deprivation caused significant decrease in the size of the prostate, seminal vesicles, bulbocavernosus/levator ani muscle complex, glans penis, and Cowper's gland with these organs shrinking to 8.6%, 6.5%, 23.9%, 54.6%, and 40.5%, respectively, of those observed in sham-operated animals (Table 2). Injection of testosterone propionate at 0.4 mg/kg increased the weight of androgen-sensitive organs 3- to 8-fold; however, testosterone injection failed to restore ventral prostate, seminal vesicles, and bulbocavernosus/levator ani muscle complex to their original size as compared with sham controls. After 10 d of treatment, oral administration of HB at 20 and 60 mg/kg failed to prevent the loss of androgen-sensitive tissue weight associated with ORX, although a slight but significant dose-dependent increase in glans penis was associated with HB treatment (55.3±1.7 mg for H₂0 and 59.6±1.6 mg for H60 versus 45.7±1.5 mg for control animals). In contrast, HB increased the weight of bulbocavernosus/levator ani muscle complex (the skeletal muscle biomarker of anabolic activity), although the change was not statistically significant. When HB was injected subcutaneously at one- and ten-fold doses relative to positive control in the Hershberger assay (testosterone propionate at 0.4 mg/kg), androgen-sensitive tissue weights did not differ from those of ORX controls with the exception of glans penis and bulbocavernosus/levator ani muscle complex, for which a significant increase was observed at 4 mg/kg HB (Table 2).

TABLE 2
Weights of androgen-sensitive tissues from sham and ORX rats treated with HB.

| Admin- | Treatment | Ventral | Seminal | Bulbocavernosus/ | Glans penis, | Cowper's |
|---|---|---|---|---|---|---|
| istration | group | prostate, mg | vesicles, mg | levator ani, mg | mg | gland, mg |
| Oral | Sham | 222.5 ± 15.2*** | 515.0 ± 12.9*** | 517.0 ± 10.5*** | 83.7 ± 0.8*** | 26.7 ± 1.8*** |
| | ORX | 19.2 ± 2.5 | 33.3 ± 3.0 | 123.7 ± 6.8 | 45.7 ± 1.5 | 10.8 ± 1.3 |
| | ORX + HB20 | 26.0 ± 2.5 | 37.3 ± 3.5 | 109.2 ± 8.6 | 55.3 ± 1.7* | 11.2 ± 0.7 |
| | ORX + HB60 | 23.0 ± 2.4 | 34.7 ± 3.3 | 137.7 ± 9.9 | 59.6 ± 1.6** | 12.5 ± 1.1 |
| | ORX + TP0.4¹ | 110.50 ± 9.5*** | 262.5 ± 12.5*** | 382.0 ± 22.0*** | 93.8 ± 4.2*** | 34.5 ± 4.7*** |
| Subcu- | ORX | 22.2 ± 3.1 | 27.8 ± 1.3 | 109.2 ± 6.4 | 41.3 ± 2.6 | 11.3 ± 0.7 |
| taneous | ORX + HB0.4 | 18.3 ± 1.2 | 29.0 ± 3.1 | 131.3 ± 7.1 | 50.7 ± 1.9 | 11.3 ± 0.5 |
| | ORX + HB4 | 23.2 ± 1.2 | 30.2 ± 1.2 | 147.5 ± 6.9* | 56.3 ± 3.3* | 10.5 ± 0.3 |
| | ORX + TP0.4 | 92.8 ± 6.3*** | 228.7 ± 35.3 | 293.8 ± 19.0*** | 72.3 ± 4.5*** | 34.3 ± 1.9*** |

Rats were fed normal diet (23.9% protein content) and gavaged daily with 20 or 60 mg/kg body weight HB or subcutaneously injected with 0.4 and 4 mg/kg body weight HB for 10 d. Results are expressed as the mean ± SEM (*P < 0.05, **P < 0.01, ***P < 0.001 when compared with ORX by one-way ANOVA and Dunnett's post-test).
¹Teststerone propionate (TP) was given as subcutaneous injection at 0.4 mg/kg and served as positive control.

In sham animals, oral administration of 20 or 60 mg/kg HB did not modify plasma testosterone levels. As expected, no plasma testosterone was detected following an oral or subcutaneous administration of HB to ORX animals that have virtually non-detectable levels of testosterone due to orchiectomy, while 0.4 mg/kg injection of testosterone propionate partially restored plasma testosterone levels in ORX rats to 20.5% of their original level (FIG. 5B).

HB showed very low androgenic activity when tested in the Hershberger assay (Table 2). Although HB produced anabolic effects in animals similar to androgens, the anabolic effects seemed to be pharmacologically different. HB administration (oral or subcutaneous) produced only minimal androgenic side effects, in sharp contrast to powerful androgenic effects of anabolic steroids. The additional observation that HB has low or no significant binding to the androgen receptor and did not modulate plasma testosterone levels (FIG. 5A) indicates that HB may exert its anabolic effect through an androgen-independent mechanism. Even though both HB and androgens contain the same steroid backbone, there are major structural differences that distinguish the two classes of compounds, including the lactone function at C6/C7, the two hydroxyls at C2 and C3, and the methyl substitution at C24. Without wishing to be bound by theory, these chemical differences may restrict HB from activating the nuclear androgen receptor and explain the difference in pharmacological responses.

### Example 8

### Physical Performance and Muscle Fiber Distribution in ORX Rats

To determine how HB affected physical performance and structural changes in muscle, animals were tested for grip strength in front and hind limbs, and for structural changes in muscle that could be detected by underlying alterations in muscle mass and function.

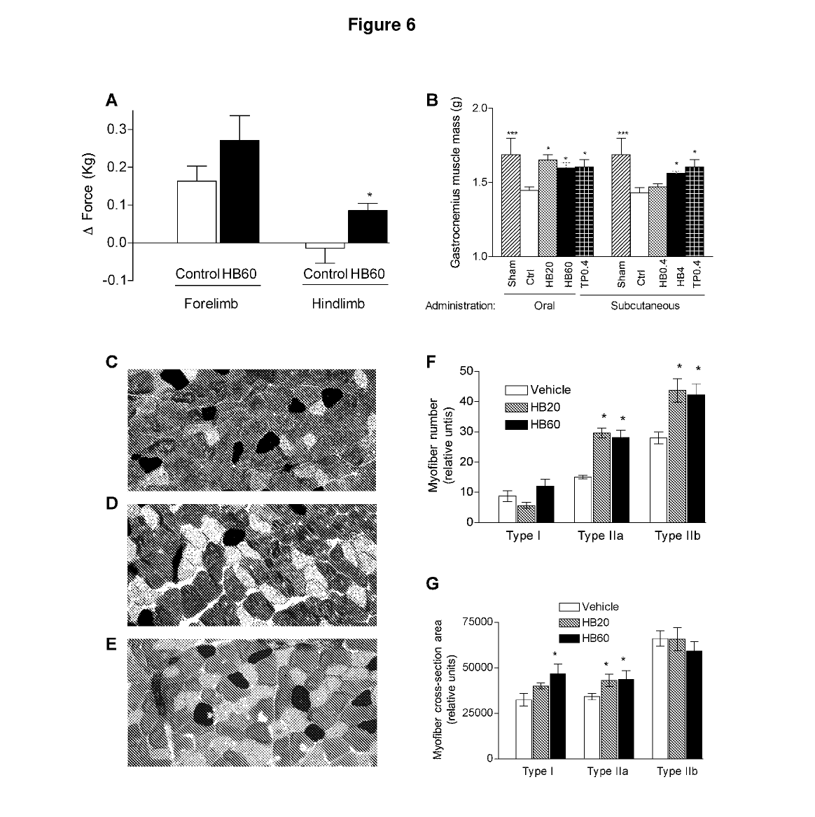

Change in grip strength of lower extremities was significantly larger in ORX animals receiving oral administration of 60 mg/kg HB per day for 10 days (0.0851±0.0197 kg versus −0.0143±0.0392 kg for controls). The change in grip strength for front limbs was also greater in HB-treated animals (FIG. 6A) but did not reach significance (0.2711±0.0660 kg versus 0.1631±0.0405 kg for controls).

As expected, androgen deprivation caused a significant decrease in the gastrocnemius muscle mass to 85.8% of that observed in sham-operated animals (FIG. 6B). Oral administration of HB to ORX rats increased gastrocnemius muscle mass by 13.8% and 10.3% in HB20 and HB60 animals, respectively, therefore almost restoring the muscle to its original size. At the same time, subcutaneous administration of HB at doses of one- and ten-fold of the positive control increased gastrocnemius muscle mass by 2.8% and 9.1%, respectively.

To determine the structural changes underlying alterations in muscle mass and function, changes in fiber distribution (FIG. 6C) and cross-section area (FIG. 6D) were analyzed. HB treatment in castrated mice prevented gastrocnemius fiber atrophy and increased median fiber area of type I and type IIa fibers above castrated control levels (P<0.001). Compared with control animals, fiber type distribution was significantly affected in HB-treated animals (20 or 60 mg/kg per day for 10 d). While the total number of type IIa and type IIb fibers increased by approximately 60% independent of HB dose, the significant increase in number of type I fibers was observed only with the higher dose of HB.

HB showed improved physical fitness of untrained ORX rats (FIG. 6). The differential effect of HB on physical fitness of front and hind limbs of untrained rats (FIG. 6), however, seems to indicate that a stronger pharmacological response was observed in the hind limb area where the abundance of androgenic receptor is typically lower in males. The 10 d oral administration of HB to castrated animals led to substantial increases in the total number of myofibers and the cross-sectional area of oxidative type I and type IIa muscle fibers important for increased physical performance and endurance. Thus, the oral administration of HB triggers a strong anabolic response with minimal or no androgenic side effects.

### Example 9

### The Effects of Position or Stereochemistry of Functional Groups on Protein Synthesis

To investigate the structure-activity relationship between functional group position or stereochemistry of HB functional groups on anabolic activity, a series of HB analogs [(22S,23S)-homocastasterone, (22S,23S)-3α-fluoro-homobrasinolide, (22S,23S)-3α-fluoro-homocastasterone, (22S,23S)-6-aza-homobrassinolide, and (22S,23S)-7-aza-homobrassinolide (compound IDs 2-6 in Table 3)] were synthesized and compared to other synthetic and naturally occurring brassinosteroids [(22R,23R)-homobrassinolide, (22S,23S)-epibrassinolide, and (22R,23R)-epibrassinolide (compound IDs 7-9 in Table 3) for their abilities to stimulate protein synthesis or inhibit protein degradation (see Example 10) in L6 rat skeletal muscle cells.

(22S,23S)-homocastasterone (compound ID 2 in Table 3) was synthesized to evaluate the influence of the C-6 lactone group on the anabolic activity of brassinosteroids. By synthesizing compounds that lack a functional hydroxyl functional group at C-2 and are fluorinated at C-3 [(22S,23S)-3α-fluoro-homobrasinolide and (22S,23S)-3α-fluoro-homocastasterone (compound IDs 3 and 4 in Table 3)], the requirement for (2α,3α)-vicinal diol moieties in the ability of brassinosteroids to promote protein accumulation in muscle cells was tested. (22S,23S)-6-aza-homobrassinolide and (22S,23S)-7-aza-homobrassinolide (compound IDs 5 and 6 in Table 3) were synthesized to contain 6-aza and 7-aza substitutions in the B ring of the brassinosteroid molecule, which allowed for the evaluation of the requirement for the 6-keto group for biological activity.

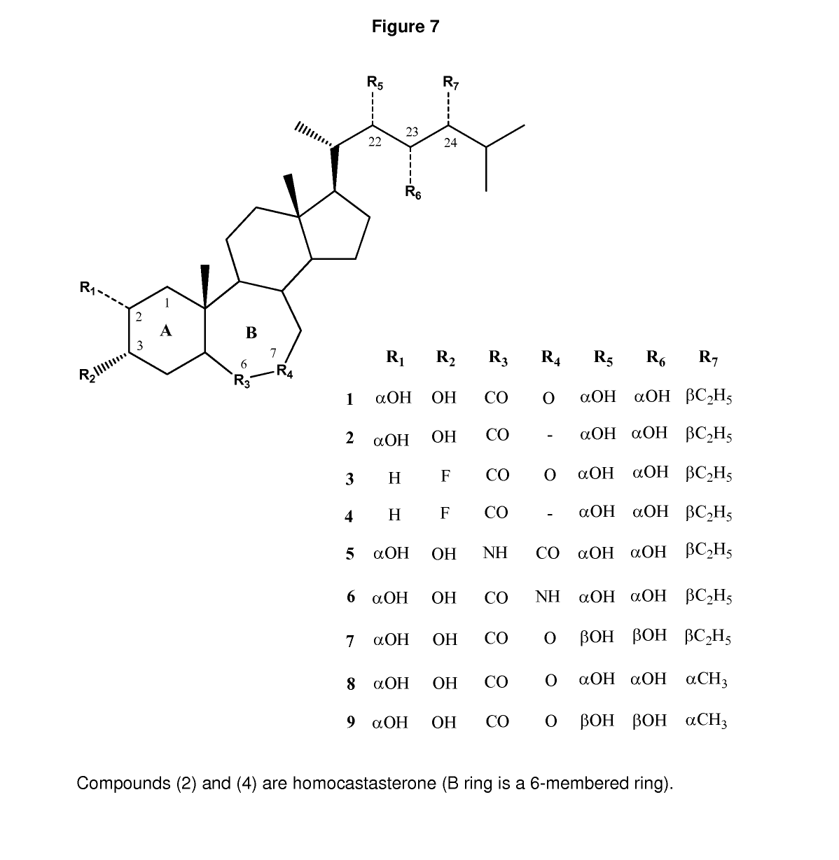

The anabolic activity of HB to other naturally occurring brassinosteroids that differ in the stereochemistry of the (22R,23R)-vicinal diol moieties (compound ID 7 in Table 3) or bear a methyl group at C-24 in the side chain of the 5α-ergostane structure (compound IDs 8 and 9 in Table 3) were also compared. The structure of HB and its analogs are shown in FIG. 7.

TABLE 3
Effect of HB (1) and its analogs (2-9) on protein accumulation in the L6 rat skeletal muscle cells.

| | | | | | Protein | Protein |
|---|---|---|---|---|---|---|
| | | | | | synthesis | degradation |
| | | | | | (% increase | (% decrease |
| ID | Common name | Chemical name | Formula | MW | over control) | over control) |
| 1 | (22S,23S)- | (22S,23S,24S)- | C29H50O6 | 494.70 | 37.2 ± 5.9*** | −24.1 ± 5.5* |
| | homobrassinolide | 2α,3α,22,23-tetrahydroxy- | | | | |
| | | B-homo-7-oxa- 5α- | | | | |
| | | cholestan-6-one | | | | |
| 2 | (22S,23S)- | (22S,23S,24S)- | C29H50O5 | 478.70 | 41.0 ± 2.7*** | −23.1 ± 4.2* |
| | homocastasterone | 2α,3α,22,23-tetrahydroxy- | | | | |
| | | 24-ethyl-5α-cholestan-6- | | | | |
| | | one | | | | |
| 3 | (22S,23S)-3α- | (22S,23S,24R)-3α-fluoro- | C29H49FO4 | 480.70 | 24.6 ± 5.3** | −20.8 ± 0.5* |
| | fluoro- | 22,23-dihydroxy-24-ethyl- | | | | |
| | homobrasinolide | B-homo-7-oxa-5α- | | | | |
| | | cholestan-6-one | | | | |
| 4 | (22S,23S)-3α- | (22S,23S,24S)-3α-fluoro- | C29H49FO3 | 464.70 | 22.5 ± 2.7** | −16.3 ± 4.2 |
| | fluoro- | 22,23-dihydroxy-24-ethyl- | | | | |
| | homocastasterone | 5α-cholestan-6-one | | | | |
| 5 | (22S,23S)-6-aza- | (22S,23S,24S)- | C29H51NO5 | 493.72 | 3.3 ± 12.4 | −13.6 ± 3.8 |
| | homobrassinolide | 2α,3α,22,23-tetrahydroxy- | | | | |
| | | 24-ethyl- B-homo-6-aza- | | | | |
| | | 5α-cholestan-7-one | | | | |
| 6 | (22S,23S)-7-aza- | (22S,23S,24S)- | C29H51NO5 | 493.72 | 20.9 ± 2.4** | −0.6 ± 2.9 |
| | homobrassinolide | 2α,3α,22,23-tetrahydroxy- | | | | |
| | | 24-ethyl- B-homo-7-aza- | | | | |
| | | 5α-cholestan-6-one | | | | |
| 7 | (22R,23R)- | (22R,23R,24S)- | C29H50O6 | 494.70 | 13.1 ± 2.4 | −14.8 ± 3.8 |
| | homobrassinolide | 2α,3α,22,23-tetrahydroxy- | | | | |
| | | B-homo-7-oxa- 5α- | | | | |
| | | cholestan-6-one | | | | |
| 8 | (22S,23S)- | (22S,23S,24R)- | C28H48O6 | 480.68 | 4.1 ± 1.7 | −5.4 ± 7.9 |
| | epibrassinolide | 2α,3α,22,23-tetrahydroxy- | | | | |
| | | 24-methyl-B-homo-7-oxa- | | | | |
| | | 5α-cholestan-6-one | | | | |
| 9 | (22R,23R)- | (22R,23R,24R)- | C28H48O6 | 480.68 | 11.7 ± 3.9 | −6.7 ± 7.9 |
| | epibrassinolide | 2α,3α,22,23-tetrahydroxy- | | | | |
| | | 24-methyl-B-homo-7-oxa- | | | | |
| | | 5α-cholestan-6-one | | | | |
| Ref | IGF-1, 6.5 nM | | | | 42.5 ± 4.5*** | — |
| Ref | Insulin, 10 nM | | | | — | −20.2 ± 1.6* |

Compounds were tested at 10 μM and results are expressed as the mean ± SEM of determinations performed in triplicate (*P < 0.05, **P < 0.01, ***P < 0.001 when compared with control by one-way ANOVA and Dunnett's post-test).

The bioactivity of HB and its analogs was evaluated by measuring increases in protein synthesis in the L6 rat skeletal muscle cells in vitro. Cells were incubated for 4 h with [³H]-phenylalanine and treated in triplicate with vehicle (0.1% ethanol) or test compound (10 μM), and protein synthesis was measured as incorporation of [³H]-phenylalanine into protein normalized by total protein (Table 3). Under these conditions, both HB and (22S,23S)-homocastasterone increased protein synthesis by 37.2±5.9% (p<0.001) and 41.0±2.7% (p<0.001), respectively. This protein synthesis increase compared favorably to the biological activity of IGF-1 at 6.5 nM (42.5±4.5%, p<0.001) that served as a positive control in this assay. Removal of the 2α-hydroxyl group and fluorination at C-3 in the A ring (compound IDs 3-4 in Table 3) led to about a 50% decrease in bioactivity (24.6±5.3%, p<0.01 and 22.5±2.7%, p<0.01, respectively) compared to the positive control. However, there was still an increase in protein synthesis. Replacement of the 7-oxalactone group with amine in the B ring (compound ID 6 in Table 3) reduced biological activity by half, while a similar replacement of the 6-carbonyl group with amine (compound ID 5 in Table 3) resulted in a complete loss of activation of protein synthesis. Modifications in the side chain (compound IDs 7-9 in Table 3) abolished the activity.

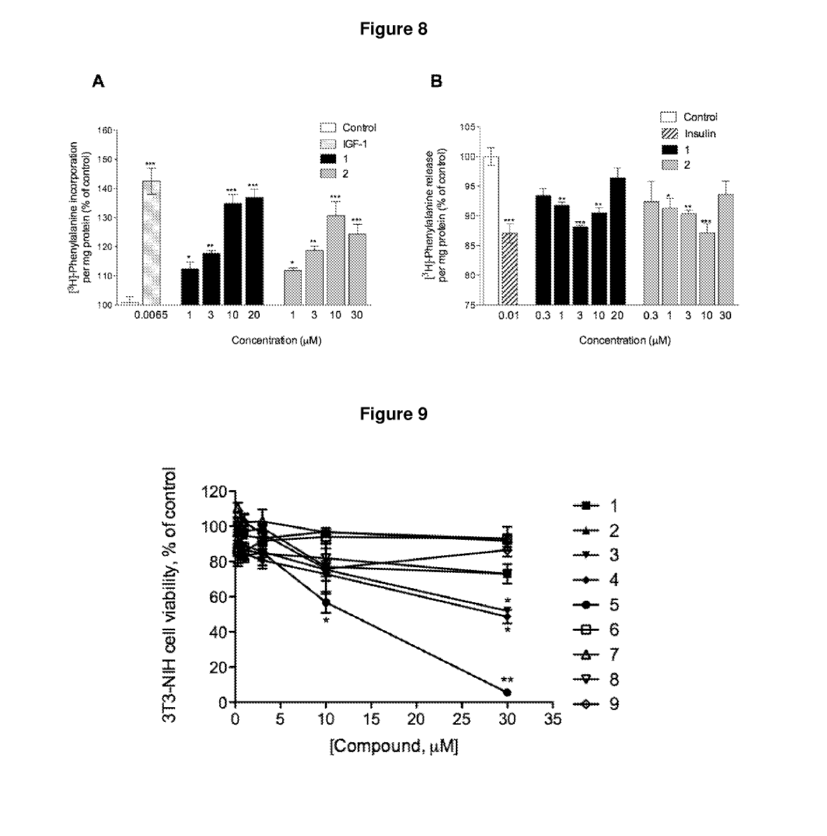

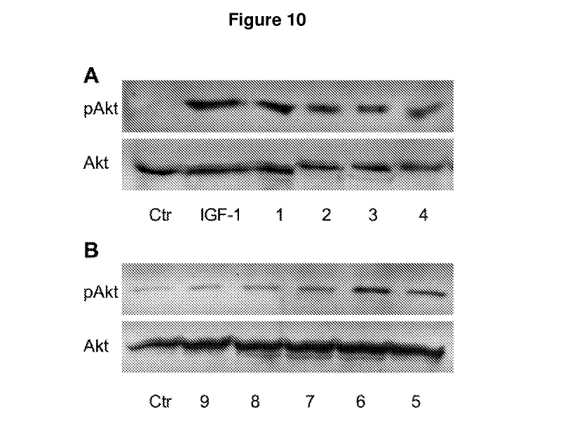

To investigate a dose-dependent effect of the most active brassinosteroids on protein synthesis, a study was carried out with 0.3-30 μM of compounds 1-2 from Table 3. Both responses approached saturation between 10 and 20 μM, with maximum increases in protein synthesis or expression of 36.9±2.9% and 40.7±4.9% (p<0.01), respectively (FIG. 8A).

A series of brassinosteroid analogs (compound IDs 2-6 in Table 3) related to HB were synthesized (FIG. 7), and the structure:activity relationships of these compounds were explored by carrying out protein synthesis and degradation assays in the L6 rat skeletal muscle cells. The results showed that (22S,23S)-homocastasterone could significantly increase protein accumulation in muscle cells similar to HB (FIG. 8). Since the only difference between these compounds is an additional 7-oxalactone group in the B ring of HB, these results indicate that a 7-oxalactone moiety is not necessary for the anabolic properties of brassinosteroids. To the contrary, moving from the lactone to the 6-ketone in plants, it was observed that brassinolide activity decreases by 50% between brassinolide and castasterone (Takatsuto et al., J. Chem. Soc. Perkin. Trans. 1:439-447, 1984). Transformation of this moiety to either 6-aza-7-oxalactone (compound ID 5, Table 3) or 6-oxo-7-aza (compound ID 6, Table 3) groups dramatically reduced their ability to stimulate protein synthesis (Table 3). These results are similar to previous studies which showed that plant brassinolide activity that was significantly reduced in 7-aza-homobrassinolide (Takatsuto et al., Chem. Pharmaceutic. Bulletin 35:211-216, 1987), while 6-aza-7-oxo-homobrassinolide was inactive (Anastasia et al., Gazzeta Chimica Italiana 114:159-161, 1984).

The effect of ring A substituents on anabolic activity of brassinosteroids was less dramatic. Replacement of the two 2α,3α-vicinal hydroxyl groups by α-fluoro group decreased but did not abolish bioactivity.

Moreover, data showed that the side chain at C-24 (methyl versus ethyl) is critically important for bioactivity in the mammalian system. Epibrassinolides (compound IDs 8 and 9 in Table 3) were found to possess very low anabolic activity in skeletal muscle cells as compared to homobrassinolides (compounds 1 and 7 in Table 3).

### Example 10

### The Effects of Position or Stereochemistry of Functional Groups on Protein Degradation

The bioactivity of HB and its analogs (as shown in Table 3) were also evaluated by measuring decreases in protein degradation in the L6 rat skeletal muscle cells in vitro. Cells were labeled overnight with [³H]-phenylalanine and subsequently treated for 4 h in triplicate with vehicle (0.1% ethanol) or test compound (10 μM), and protein degradation was assessed as release of acid-soluble [³H]-phenylalanine into the media normalized by total protein (Table 3). HB, (22S,23S)-homocastasterone, (22S,23S)-3α-fluoro-homobrasinolide, (22S,23S)-3α-fluoro-homocastasterone, (22S,23S)-6-aza-homobrassinolide, and (22R,23R)-homobrassinolide) reduced protein degradation in vitro, but the potency of their activities differed according to their structure. Compound IDs 1-3 showed the strongest prevention of protein degradation, by more than 20%, which compared favorably with 10 nM insulin treatment that served as a positive control in this assay (20.2±1.6%, p<0.05). Prevention of degradation was dependent on the presence of the ethyl group at the C-24 position in the side chain (comparing compounds 1 and 7 versus compounds 8 and 9) and was partially dependent on the stereochemistry of the (22R,23R)-vicinal diol moieties (comparing compounds 1 and 7). Interestingly, replacement of the 7-oxalactone group with amine in the B ring of (22S,23S)-7-aza-homobrassinolide completely abolished its effect on protein degradation, while a similar replacement of the 6-carbonyl group with amine in (22S,23S)-6-aza-homobrassinolide had only a minor effect on its biological activity.

Among the most active compounds in this assay, HB at concentrations of 0.3-20 μM inhibited protein degradation dose-dependently and HB activity reached a plateau between 3 and 10 μM (FIG. 8B). At a lower concentration, 1 μM HB decreased protein degradation by 8.2±0.6% above control levels (p<0.05). Compound 2 at concentrations of 0.3-30 μM inhibited protein degradation dose-dependently and activity of compound 2 reached a plateau at 10 μM. At the lower concentration, compound 2 at 1 μM suppressed protein degradation by 8.7±1.7% above control levels (p<0.05).

These studies provide evidence that the 6-keto group and the 22α,23α-hydroxyls are critical for anabolic activity of brassinosteroids in rat skeletal muscle cells. Such information is useful for the design of novel therapeutic molecules possessing high anabolic selectivity. In addition, (22S,23S)-homobrassinolide and (22S,23S)-homocastasterone, were confirmed to possess the greatest anabolic activity among the molecules analyzed.

### Example 11

### Cytotoxicity in L6 Muscle Cells and 3T3 Fibroblast Cells

All brassinosteroids and their analogs showed no toxicity in fully differentiated L6 rat skeletal myotubes when administered in vitro to the cells at concentrations up to 30 μM as established by the MTT assay and cytological observations. Therefore, all compounds were tested in a standard test for basal cytotoxicity using a 3T3/NIH murine fibroblast cell culture. (22S,23S)-6-aza-homobrassinolide was the only brassinosteroid analog that inhibited cell proliferation in a dose-dependent manner with a half-maximal inhibitory concentration(IC₅₀) of 12.5 μM. Fluorination at C-3 in the A ring ((22S,23S)-3α-fluoro-homobrasinolide and (22S,23S)-3α-fluoro-homocastasterone) led to increased cytotoxicity as compared to the original brassinosteroids ((22S,23S)-homobrassinolide and (22S,23S)-homocastasterone) (FIG. 9).

### Example 12

### AKT Phosphorylation

Because AKT modulates intracellular targets known to regulate protein synthesis and degradation, an experiment was carried out to determine the effect of brassinosteroids on AKT phosphorylation. Consistent with the results obtained with the [³H]-phenylalanine incorporation assay, bioactive brassinosteroids stimulated phosphorylation of AKT in rat skeletal muscle cell culture (FIG. 8).

Both HB and (22S,23S)-homocastasterone treatments resulted in significant activation of AKT after 1 h, a much slower response than that produced by IGF-1, which phosphorylates AKT within 10 min. A similar delayed AKT response has been reported previously for ecdysteroids (Gorelick-Feldman et al., J. Agric. Food Chem. 56:3532-3537, 2008).

### Example 13

### Pharmacogenomic Effect of HB In Vivo

As set out above herein, HB treatment (60 mg/kg body weight daily to healthy rats fed a normal diet for 24 d) was associated with a 14.2% increase in lean body mass and improved physical fitness in untrained rats. In order to determine how HB was causing these effects, the pharmacogenomic properties of HB were studied in healthy rats by examining changes in gene expression after oral administration of HB (60 mg/kg for 24 d).

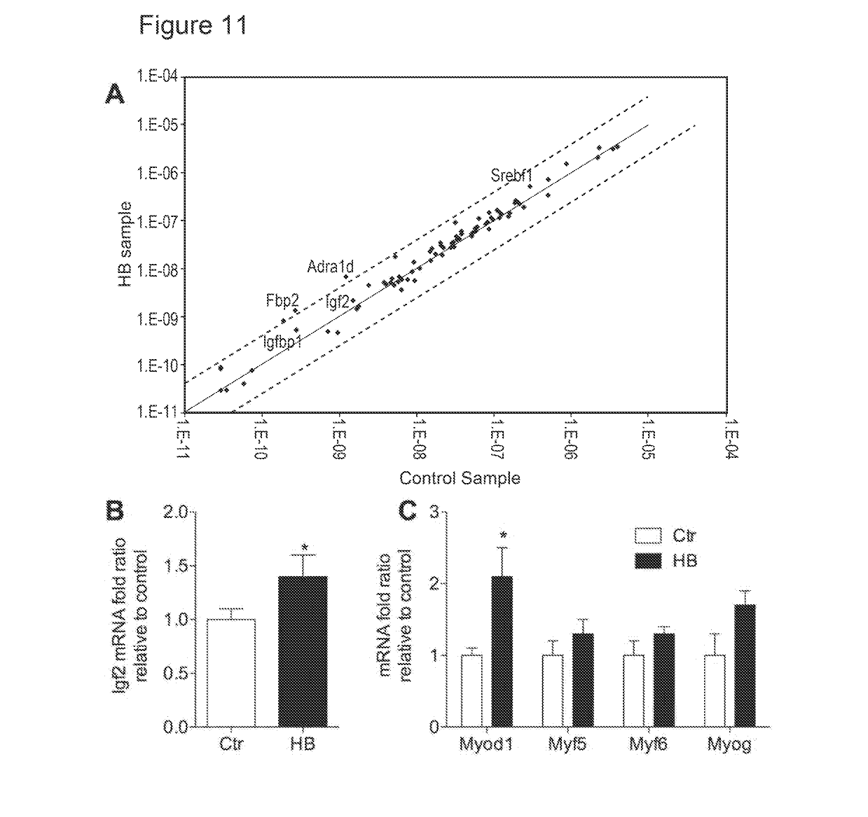

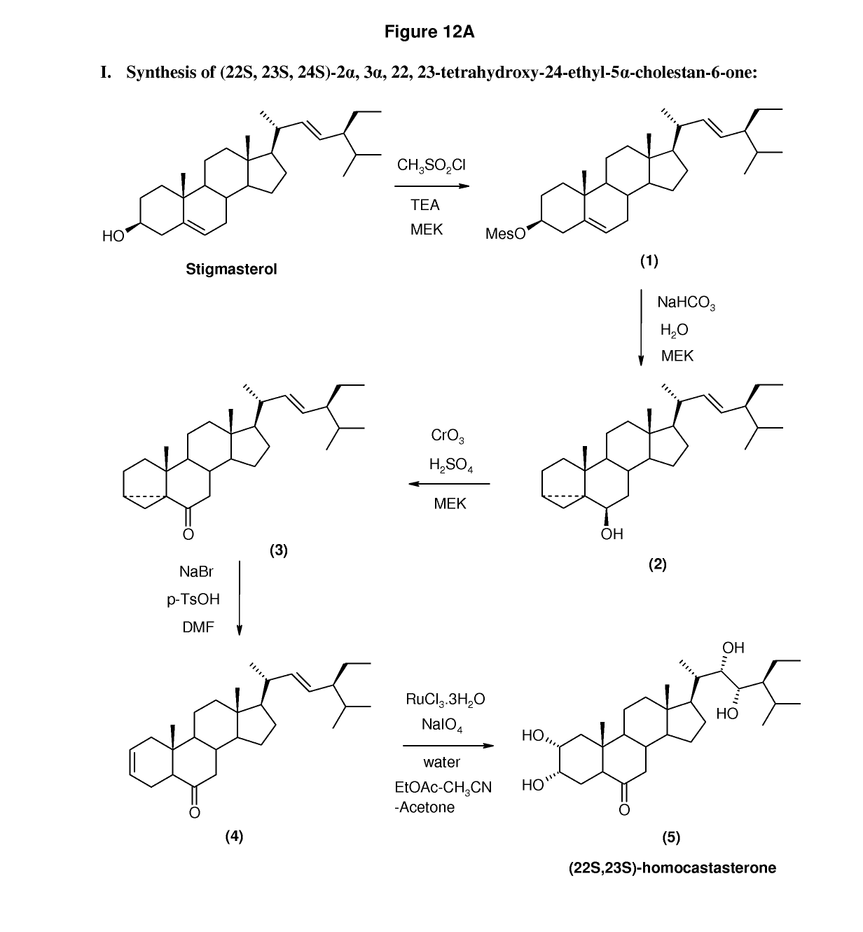

Pooled RNA samples obtained from frozen skeletal muscle (gastrocnemius muscle biopsies of vehicle- (Ctr) and HB-treated animals) were used in a rat insulin signaling PCR array to measure mRNA response to HB. Of the 84 genes responsible for insulin signaling, the phosphoinositide 3-kinase (PI3K) and mitogen-activated protein kinase (MAPK) pathways, carbohydrate metabolism, and cell cycle regulation, two subsets of genes were expressed more greatly in the HB-treated group than in the Ctr group. However, the magnitude of the differences varied (FIG. 11A).

The first subset of upregulated genes included a set of target genes upregulated through the PI3K/AKT signaling pathway: alpha-1D adrenergic receptor (ADRA1D) (6.5-fold), insulin-like growth factor binding protein 1 (IGFBP1) (2.5-fold), and sterol regulatory element-binding protein 1 (SREBP-1) (2.5-fold). The second subset contained upregulated genes that regulate muscle cell growth and carbohydrate metabolism: fructose-1,6-bisphosphatase 2 (FBP2) (4.5-fold) and IGF-2 (1.4-fold).

The alpha-1D adrenergic receptors mediate endogenous functions of catecholamines, which involve coupling to G proteins followed by activation of phospholipase Cβ and protein kinase C (Strosberg, Obes. Res. 3 Suppl 4:501 S-505S, 1995). Messenger RNA levels of IGF-2 and IGFBP1 were also upregulated in skeletal muscle of rats administered HB. Insulin-like growth factor 2 (IGF-2) expression during skeletal muscle differentiation is regulated at the transcriptional level (Kou et al., Mol. Endocrinol. 7:291-302, 1993), and signaling through the insulin-like growth factor 1 (IGF-1) receptor by locally produced IGF-2 defines a pathway that is critical for normal muscle growth and regeneration.

These results were further verified for IGF-2 gene expression by using RT-PCR on individual muscle samples from Ctr and HB-treated animals (FIG. 11B). No changes in expression of eukaryotic translation initiation factor 2B1 (EIF2B1) were noted.

Even though inducible activation of AKT is sufficient to increase skeletal muscle mass and force without satellite cell activation (Blaauw et al., FASEB J 23:3896-3905, 2009), the effect of HB treatment on the expression of various myogenic transcription factors that modulate muscle growth and differentiation was also evaluated. HB treatment induced the upregulation of myogenic differentiation 1 (MYOD1) (2.1-fold), myogenic factor 5 (MYF5) (1.3-fold), myogenic factor 6 (MYF6) (1.3-fold), and myogenin (also known as myogenic factor 4 or MYOG) (1.7-fold) (FIG. 11C). MYOD1 was the only transcription factor associated with a greater than 2-fold upregulation following HB treatment (FIG. 11C). MYOD1 induces cell differentiation by activating muscle-specific genes and it is important in the switch from cellular proliferation to differentiation (Solomon et al. J. Endocrinol. 191:349-360, 2006).

The results indicate that HB potently stimulated two sets of genes involved in muscle cell growth and carbohydrate metabolism. Among the genes that are upregulated, ADRA1D showed the most notable 6.5-fold induction (FIG. 11A). The al adrenergic receptors mediate endogenous functions of catecholamines, which involve coupling to G proteins followed by activation of phospholipase Cβ and protein kinase C.

Without wishing to be bound by theory, it may be that ADRA1D may regulate muscle survival and differentiation (Saini et al., Cell. Physiol. Biochem. 25:253-262, 2010). Messenger RNA levels of IGF-2 and IGFBP1 were also upregulated in skeletal muscle of rats administered HB. IGF-2 expression during skeletal muscle differentiation is regulated at the transcriptional level (Kou et al., Mol. Endocrinol. 7:291-302, 1993), and signaling through the Insulin-like growth factor 1 (IGF-1) receptor by locally produced IGF-2 defines a pathway that is significant in normal muscle growth and regeneration.

### Example 14

### Brassinosteroid Treatment Decreased Wound Closure Time

To evaluate the potential anabolic effects of plant brassinosteroids on the skin of animals, in vitro scratch wound closure experiments were carried out.

3T3 Swiss albino mouse fibroblast cells were obtained from ATCC (Manassas, Va.). Cell lines were routinely passaged every 3-4 days and maintained in DMEM containing 10% FBS and 0.1% penicillin-streptomycin at 37° C. and 5% CO₂. Cells were subcultured in 24-well dishes for cell proliferation and scratch wound closure assays, and in 96-well dishes for cell viability and nitric oxide production studies.

3T3 fibroblasts were seeded into 24-well dishes at a concentration of 3×10⁵ cells/ml and cultured to nearly confluent cell monolayers. On the day of the experiment, a linear wound was generated in the monolayer with a sterile 100 μl plastic pipette tip and any cellular debris was removed by washing cells once with sterile PBS. Fresh DMEM medium containing vehicle (0.1% ethanol), positive control (0.5% FBS), or various concentrations of the fractions, sub-fractions or pure compounds was added to a set of 3 wells per dose and incubated for 12 h at 37° C. with 5% CO₂. Cells were then visualized with 20 μl of 10% methylene blue in PBS for 5 min. Three representative images of the scratched areas from each well were photographed at 0 and 12 h to estimate the scratch wound closure. Images were analyzed using ImageJ program (NIH, Bethesda, Md.) and % wound closure was calculated relative to vehicle control and was reported as wound closure % over control (Table 4).

Treatment with HB and its analogs improved wound closure in vitro. See Table 4.

TABLE 4
Effect of HB (1) and its analogs (2-9) on scratch wound closure in 3T3 cells.

| | | | | | Wound Closure |
|---|---|---|---|---|---|
| ID | Common name | Chemical name | Formula | MW | (% over control) |
| 1 | (22S,23S)- | (22S,23S,24S)- | C29H50O6 | 494.70 | 30.2 ± 11.2* |
| | homobrassinolide | 2α,3α,22,23-tetrahydroxy- | | | |
| | | B-homo-7-oxa-5α- | | | |
| | | cholestan-6-one | | | |
| 2 | (22S,23S)- | (22S,23S,24S)- | C29H50O5 | 478.70 | 32.9 ± 14.1* |
| | homocastasterone | 2α,3α,22,23-tetrahydroxy- | | | |
| | | 24-ethyl-5α-cholestan-6- | | | |
| | | one | | | |
| 3 | (22S,23S)-3α- | (22S,23S,24R)-3α-fluoro- | C29H49FO4 | 480.70 | 15.3 ± 2.6 |
| | fluoro- | 22,23-dihydroxy-24-ethyl- | | | |
| | homobrasinolide | B-homo-7-oxa-5α- | | | |
| | | cholestan-6-one | | | |
| 4 | (22S,23S)-3α- | (22S,23S,24S)-3α-fluoro- | C29H49FO3 | 464.70 | 27.7 ± 4.0* |
| | fluoro- | 22,23-dihydroxy-24-ethyl- | | | |
| | homocastasterone | 5α-cholestan-6-one | | | |
| 5 | (22S,23S)-6-aza- | (22S,23S,24S)- | C29H51NO5 | 493.72 | Not quantified yet |
| | homobrassinolide | 2α,3α,22,23-tetrahydroxy- | | | |
| | | 24-ethyl-B-homo-6-aza- | | | |
| | | 5α-cholestan-7-one | | | |
| 6 | (22S,23S)-7-aza- | (22S,23S,24S)- | C29H51NO5 | 493.72 | 27.3 ± 3.6* |
| | homobrassinolide | 2α,3α,22,23-tetrahydroxy- | | | |
| | | 24-ethyl-B-homo-7-aza- | | | |
| | | 5α-cholestan-6-one | | | |
| 7 | (22R,23R)- | (22R,23R,24S)- | C29H50O6 | 494.70 | Not quantified yet |
| | homobrassinolide | 2α,3α,22,23-tetrahydroxy- | | | |
| | | B-homo-7-oxa-5α- | | | |
| | | cholestan-6-one | | | |
| 8 | (22S,23S)- | (22S,23S,24R)- | C28H48O6 | 480.68 | Not quantified yet |
| | epibrassinolide | 2α,3α,22,23-tetrahydroxy- | | | |
| | | 24-methyl-B-homo-7-oxa- | | | |
| | | 5α-cholestan-6-one | | | |
| 9 | (22R,23R)- | (22R,23R,24R)- | C28H48O6 | 480.68 | Not quantified yet |
| | epibrassinolide | 2α,3α,22,23-tetrahydroxy- | | | |
| | | 24-methyl-B-homo-7-oxa- | | | |
| | | 5α-cholestan-6-one | | | |

Compounds were tested at 10 μM and results are expressed as the mean ± SEM of determinations performed in triplicate (*P < 0.05 when compared with control by one-way ANOVA and Dunnett's post-test).

Twelve C57BL/6J male mice (20±4 g; Charles River Laboratories, MA) were housed in individual chambers in a room maintained at a constant temperature with a 12 h light-dark cycle. Animals had free access to food and water. Animals were allowed to adapt to new conditions for seven days. Animals were handled daily to reduce the stress of physical manipulation. Animals were randomized into groups according to body weight one day prior to dosing.

Under volatile anesthesia (5% isoflurane to effect), the shoulder and back region of each animal was shaved. A sharp punch (ID 6 mm) over their lumbar spine was applied to remove the skin, including panniculus carnosus and adherent tissues. Test substances were administered topically (TOP), immediately following cutaneous injury, once daily for 10 consecutive days. Substances used were (1) Homobrassinolide (10 μg/mouse), (2) positive control (CGS-21680, a specific adenosine A_{2A} subtype receptor agonist, 10 μg/mouse,), or (3) vehicle (1.5% carboxymethyl cellulose (CMC)).

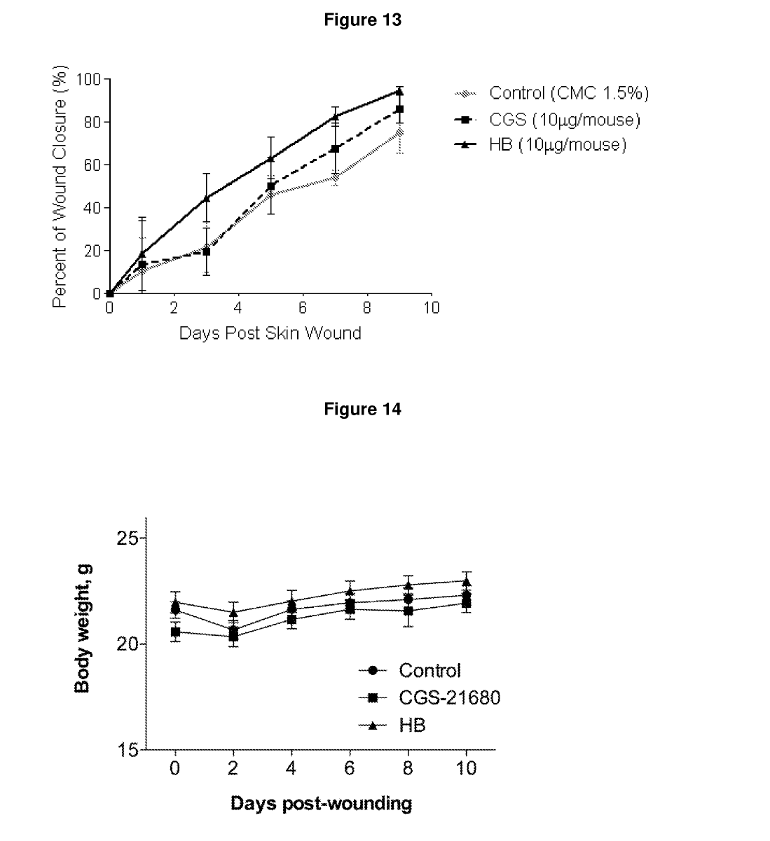

To investigate the kinetics of wound healing, wound size was measured on days 1, 3, 5, 7, and 9 following treatment. Wounds were traced onto clear plastic sheets and were measured by use of an ImageJ program (rsbweb.nih.gov/ij/), online from the National Institutes of Health (NIH). ImageJ is a public domain, Java-based image processing program developed at the NIH. Time to wound closure was estimated by comparing the area of the wound undergoing treatment to the areas of the wound undergoing control treatments. The percent closure of the wound (%) was calculated, and wound half-closure time (the time at which 50% of the cutaneous wound is closed, CT50) was analyzed by linear regression using Graph-Prism (Graph Software USA). At the end of experiment (10 days after the induction of the wound), animals were euthanized by CO₂ gas inhalation. Blood was collected by heart puncture. Wound tissue and muscle were collected for immunohistochemical analyses and wound healing factor assays. HB increased the percent of wound closure in vivo in a mouse model of wound closure (FIG. 13). Thus, BR treatment shortened, i.e. decreased, the time necessary for wound healing and, thus, had a beneficial effect on skin in the treatment of wounds.

### Example 15

### Brassinosteroids Increased Elastin Production in Skin Cells

To evaluate the potential anabolic effects of plant brassinosteroids on the skin of animals, elastin gene expression in human dermal fibroblasts was measured in response to treatment with homocastasterone.

Human neonatal dermal fibroblasts (HNDF) were maintained in DMEM+10% FBS and were kept in a humidified 37° C. incubator with 5% CO₂. Cells were plated at a density of 1×10⁴ cells per well in a 24-well plate and incubated for 24 h, or until 100% confluency was reached. Subsequently, the media was removed and cells were washed with 500 μL PBS twice to remove detached cells or cell debris. HNDF were then treated for 16 h as follows: DMEM only, retinoic Acid 5 μM, retinoic acid 10 μM, Homocastasterone 5 μM and Homocastasterone 10 μM.

RNA was extracted from HNDF using Trizol reagent (Invitrogen) following manufacturer instructions. RNA was quantified spectrophotometrically by absorbance measurements at 260 nm and 280 nm using the NanoDrop system (NanoDrop Technologies, Wilmington, Del.). Quality of RNA was assessed by separation in gel-electrophoresis. To remove any traces of DNA contamination, RNA was then treated with Dnase I (Invitrogen) following the manufacturer guidelines. Complementary DNAs (cDNAs) were synthesized using 3.0 μg of RNA for each sample using Applied Biosystems High Capacity cDNA Reverse Transcription Kit following the manufacturer's protocol.

Quantitative RT-PCR (qPCR) amplifications were carried out in triplicate on an ABI 7300 Real-Time Detection System in a total volume of 25 μl containing 12.5 μl Brilliant SYBR® Green PCR master mix (Applied Biosystems), 5 μl of the 1:25 diluted cDNA, 0.5 μl of 6 μM gene-specific primers (IDT, Coralville, Iowa) and 7 μl PCR-grade water. Primers were synthesized by Integrated DNA Technologies, Inc. (Coralville, Iowa, USA) as follows: β-actin, forward primer 5′-ACG TTG CTA TCC AGG CTG TGC TAT-3′ (SEQ ID NO: 1), reverse primer 5′-CTC GGT GAG GAT CTT CAT GAG GTA GT-3′ (SEQ ID NO: 2); elastin, forward primer 5′-AAG CAG CAG CAA AGT TCG GT-3′ (SEQ ID NO: 3), and reverse primer 5′-ACT AAG CCT GCA GCA GCT CCA TA-3′ (SEQ ID NO: 4). qPCR amplifications were performed on the 7300 Real Time PCR System (A&B Applied Biosystems) using 1 cycle at 50° C. for 2 min, 1 cycle at 95° C. for 10 min, followed by 40 cycles of 15 s at 95° C. and 1 min at 60° C. The dissociation curve was completed with one cycle of 15 s at 95° C., 1 min at 60° C., and 15 s of 95° C. No RT (NRT) and no template control (NTC) were included in each experiment as quality control steps. Target mRNA expression was analyzed using the ΔΔCt method and normalized with respect to the expression of the β-actin housekeeping gene. Cells treated with DMEM only (negative control) served as the calibrator sample in this study, and a value of 1.0 was assigned to the target gene expression of the calibrator sample. All samples were run in triplicate. A p value of 0.001 was considered to be significant.

TABLE 5
Homocastasterone increases elastin gene expression in human
dermal fibroblasts.

| | Increase in elastin | |
|---|---|---|
| | mRNA levels | |
| Compound | (-fold over control) | SD |
| Retinoic Acid 5 μM | 2.9** | 0.1 |
| Retinoic Acid 10 μM | 2.7** | 0.2 |
| Homocastasterone 5 μM | 1.7 | 0.3 |
| Homocastasterone 10 μM | 3.2** | 0.4 |

**p < 0.001, One-way ANOVA, Bonferroni's Multiple Comparison Test, n = 3

Homocastasterone significantly increased mRNA levels of elastin in HNDF. In fact, homocastasterone's effect in increasing elastin mRNA level was comparable to the effect of retinoic acid (positive control) and up to 3.23±0.4-fold higher than that of non-treated cells (Table 5). Thus, homocastasterone has a positive effect on elastin production in skin. This experiment indicates that BRs, like homocastasterone, demonstrate utility in the treatment of skin, and are useful as a topical anti-aging, skin care product.

### Example 16

### Anabolic Effects of HB on Skin

To evaluate the potential anabolic effects of plant brassinosteroids on the skin of animals, 20 and 60 mg/kg body weight of HB (HB20 and HB60, respectively) are orally administered daily to healthy rats fed normal diet for 24 d.

Collagen and elastin production are measured in the skin of animals treated with HB versus their control counterparts. Protein degradation of collagen and elastin is measured in the skin of animals treated with HB versus their control counterparts, e.g., by collagen or elastin ELISAs. The amount of collagen and elastin protein present in the skin of animals treated with HB is measured versus their control counterparts.

Treatment with HB increases collagen and elastin production in skin, decreases the protein degradation of collagen and elastin in skin, and/or increases the content of collagen and elastin in skin.

### Example 17

### Animal Model of Wound Healing

To evaluate the effect of plant brassinosteroids on wound healing, an animal model was used in various aspects as described herein below.

Twenty seven six-week-old male C57BL/6J mice, obtained from the Jackson Laboratory (Bar Harbor, Me.), were housed in individual chambers in a room maintained at a constant temperature with 12 h light-dark cycle. Mice had free access to food and water. Mice were allowed to adapt to new conditions for seven days and handling the mice was performed daily during this time to reduce the stress of physical manipulation. Mice were randomized into groups (n=9) according to body weight one day prior to dosing. Under volatile anesthesia (5% isoflurane to effect), the shoulder and back region of each animal was shaved. A sharp punch (ID 6 mm) over lumbar spine was applied to remove the skin including panniculus carnosus and adherent tissues. Test substance (control vehicle 1.5% carboxymethyl cellulose, or 10 μg/mouse of either HB or the adenosine receptor agonist CGS-21680 (Valls et al., Biochem. Pharmacol. 77: 1117-1124, 2009) as a positive control) was administered topically, immediately following cutaneous injury, and then daily for 10 days.

To investigate the kinetics of wound healing, wound size was photographed and measured every two days with ImageJ software (rsbweb.nih.gov/ij/). Time to wound closure was estimated by comparing the area of treatment wounds to the area of control wounds. The percent closure of the wound (%) was calculated, and time at which 50% of the cutaneous wound was closed (CT50) was analyzed by linear regression. At the end of experiment, animals were euthanized by CO₂ gas inhalation. Wounded tissue samples were collected by snap-freezing in liquid nitrogen and stored at −80° C. for wound healing factor assays or fixed in 4% paraformaldehyde for routine histological sectioning and staining using Mayers haematoxylin and eosin.

### Example 18

### Effect of Brassinosteroid Treatment on Body Weight and Food Intake in an Animal Model of Wound Healing

To evaluate the effect of wounding on body weight and food intake, animals were wounded and treated for 10 days with HB, CGS-21680 (positive control) or vehicle (negative control) according to the protocol described in Example 17. Basically, a sharp punch over the lumbar spine was applied to remove the skin and then treatment (10 μg/mouse of either HB, CGS-21680, or vehicle) was administered topically for 10 days. Body weight and food intake were measured in the animals every other day for 10 days after wounding.

Data were represented as mean±SEM. Statistical analyses were performed with GraphPad Prism 4.0 (San Diego, Calif.) using one-way ANOVA completed by a multicomparison Dunnett's test. Wound closure associated body weight change was analyzed by two-factor repeated-measures ANOVA with time and treatment as independent variables. P-values of less than 0.05 were considered significant.

While there were no overall significant effects for body weight in the 10-day period following wounding, all mice lost weight on day 2 post-wounding, with weight gain resuming on day 4 (FIG. 14). Mice lost 1.5 g of body weight by day 2, and there was no significant difference between the treatments, although there was a tendency for HB to reduce weight loss associated with injury. There was also a transient reduction in food intake that lasted for 48 h post-wounding with no significant differences noted between the treatments.

### Example 19

### HB Treatment Improves Wound Healing

To evaluate the effect of plant brassinosteroids on cutaneous wound healing, HB (10 μg/mouse/day), positive control (CGS-21680) (10 μg/mouse/day), or negative control (vehicle alone) was administered topically daily for 10 days after wounding as described in detail in Example 17. Wound sizes were photographed and measured every 2 days for 10 days. Wound closure (%) relative to day 1 was determined every 2 days, and CT50 was calculated by linear regression. Two-factor repeated-measures ANOVA were performed. P-values of less than 0.05 (n=9) were considered significant.

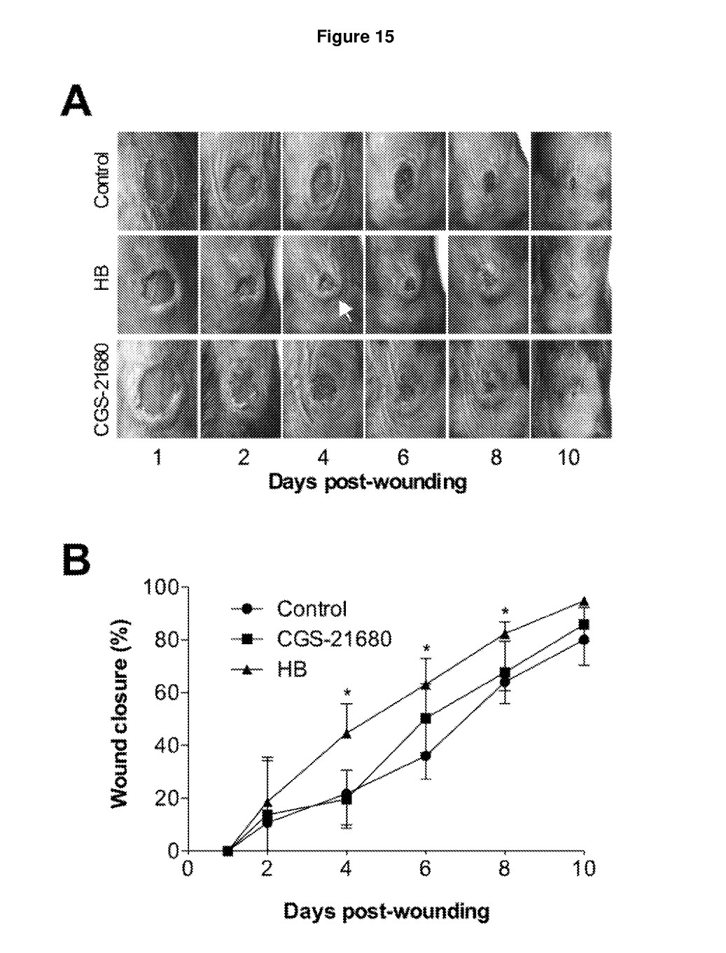

Cutaneous wound healing was significantly improved in animals receiving HB compared controls treated with vehicle alone (FIG. 15A). The brassinosteroid effect appeared to occur in the early phases (up to day 6 post-wounding) of wound healing (FIG. 15B). CT50, was significantly reduced by both HB (5.4±0.3 days) and positive control CGS-21680 (6.2±0.4 days) compared with vehicle controls (7.2±0.2 days). The strongest effect associated with HB treatment was observed on day 4, when inflammatory and tissue repair stages overlap. Without being bound by theory, it is possible that HB promotes wound healing by stimulation of cell proliferation or migration into the wound area.

When wound data were expressed in terms of percent of original wound size, there was a 2-fold increase in speed of wound closure relative to control mice. Another interesting morphological observation associated with HB treatment was increased volume of the wound edges that reached prominence on day 4 and slowly subsided on days 6-8 to completely disappear on day 10 post-wounding. This effect was absent in both negative and positive controls.

### Example 20

### HB Reduces Proinflammatory Markers in Healing Wounds

Cutaneous would healing is characterized by an initial inflammatory response. To evaluate the effect of brassinosteroids on inflammation in wound healing, mRNA levels of proinflammatory cytokines TNF-α and TGF-β and an adhesion chemokine ICAM-1 were measured in the wound tissue of control and treated mice on day 10 post-wounding and treatment with HB, positive control (CGS-21680), or negative control (vehicle).

### Real-Time Quantitative PCR

Total RNA was extracted from fibroblasts using Trizol (Invitrogen, Carlsbad, Calif.). RNA was quantified spectrophotometrically by absorbance measurements at 260 and 280 nm using the NanoDrop reader (NanoDrop Technologies, Wilmington, Del.). Quality of RNA was assessed by gel electrophoresis. RNA was then treated with DnaseI (Invitrogen, Carlsbad, Calif.) to remove traces of DNA contamination and the cDNAs were synthesized with 2.5 μg of RNA using Stratascript reverse transcriptase (Stratagene, Santa Clara, Calif.) according to the manufacturers' protocols. Quantitative PCR was performed in duplicate essentially as described previously (Komarnytsky et al., Int. J. Obes. 35: 236-43, 2011) using the following gene-specific primers (IDT, Coralville, Iowa) selected using the Primer Express 2.0 software (Applied Biosystems, Foster City, Calif.): β-actin, forward primer 5′-GGG AAA TCG TGC GTG ACA TT-3′ (SEQ ID NO: 5), and reverse primer 5′-GCG GCA GTG GCC ATC TC-3′ (SEQ ID NO: 6). Target gene expression of the housekeeping gene β-actin was assigned a value of 1. Samples were subjected to a melting curve analysis to confirm the amplification specificity. The relative change in the target gene β-actin with respect to the endogenous control gene was determined using 2ΔΔCT method (Winer et al., Anal. Biochem. 270: 41-9, 1999).

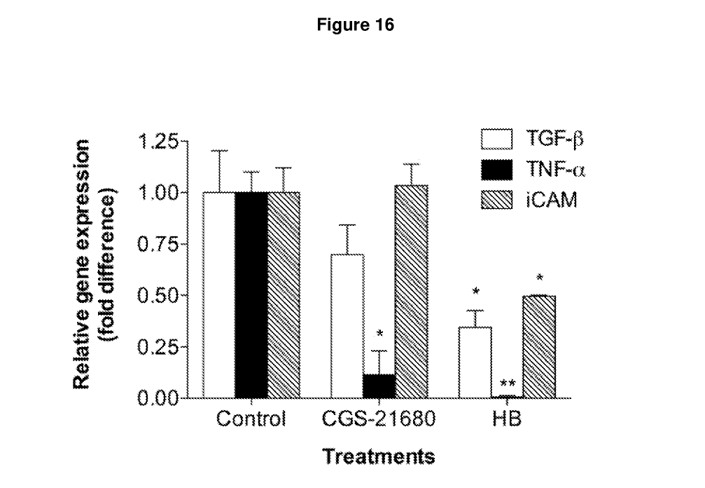

HB treatment was associated with a weak effect on the downregulation of TGF-β mRNA, significant suppression of ICAM-1 mRNA, and nearly complete downregulation of TNF-α mRNA (FIG. 16). Wound tissue from animals treated with the adenosine receptor agonist CGS-21680 (positive control) showed a remarkable suppression of TNF-α mRNA, but no effect on either TGF-β or ICAM-1 mRNA levels.

### Example 21

### Effect of Brassinosteroids on Cell Viability and Proliferation

To further elucidate effects of brassinosteroid treatment on cell proliferation and determine the structure-activity requirements for brassinosteroid biological activity, the cytotoxic and cell proliferation effects of HB (FIG. 1) and its natural or synthetic analogues (FIG. 7, Table 6, and Esposito et al., J. Med. Chem. 54: 4057-66, 2011) were analyzed in 3T3 mouse fibroblast cell culture.

### Cell Culture

An NIH 3T3 murine embryonic fibroblast cell line CCL-92 (“3T3 fibroblasts”) was obtained from ATCC (Manassas, Va.). Cells were routinely maintained in Dulbecco's modified Eagle's medium (DMEM) containing 10% FBS and 0.1% penicillin-streptomycin at 37° C. and 5% CO₂ and passaged every 3-4 days. Cells were subcultured into 96-well plates for proliferation and cell viability assays, and 24-well plates for scratch wound closure studies (Greiner Bio One, Monroe, N.C.).

### Cell Viability and Proliferation Assays

3T3 fibroblasts were seeded in a 96-well flat bottom plate at a density of 1×10⁴ cells/well. Cell viability was measured by an MTT (3-(4,5-Dimethylthiazol-2-yl)-2,5-diphenyltetrazolium bromide) assay in triplicate (0.3-30 μM of test substance for 4 h) essentially as described by Mosmann (J. Immunol. Methods 65, 55-63, 1983) and quantified spectrophotometrically at 550 nm using a microplate reader (Molecular Devices, Sunnyvale, Calif.). Concentrations of test reagents that showed no changes in cell viability compared with that of the vehicle (0.1% ethanol) were selected for further studies. For cell proliferation studies, cells were treated in triplicate with 0.1-10 μM of test substance for 24 h and assayed using a BrdU (5-bromo-2′-deoxyuridine) kit from Amersham (Uppsala, Sweden).

HB [(22S,23S,24S)-2α,3α,22,23-tetrahydroxy-24 ethyl-β-homo-7-oxo-5α-cholestane-6-one] was purchased from Waterstone Technology (Carmel, Ind.) and its structure was confirmed by ESI-LCMS and NMR. Brassinosteroid analogues 2-9 (FIG. 7), including homocastasterone (22S,23S,24S)-2α,3α, 22,23-tetrahydroxy-24-ethyl-5α-cholestan-6-one (2), (22S,23S,24S)-3α-fluoro-22,23-dihydroxy-24-ethyl-B-homo-7-oxa-5α-cholestan-6-one (3), (22S,23S,24S)-3α-fluoro-22,23-dihydroxy-24-ethyl-5α-cholestan-6-one (4), (22S,23S,24S)-2α,3α-22,23-tetrahydroxy-24-ethyl-B-homo-6-aza-5α-cholestan-7-one (5), (22S,23S,24S)-2α,3α-22,23-tetrahydroxy-24-ethyl-B-homo-7-aza-5α-cholestan-6-one (6), (22R,23R,24S)-2α,3α-22,23-tetrahydroxy-24-ethyl-B-homo-7-oxa-5α-cholestan-6-one (7), (22S,23S,24R)-2α,3α-22,23-tetrahydroxy-24-methyl-B-homo-7-oxa-5α-cholestan-6-one (8), and (22R,23R,24R)-2α,3α-22,23-tetrahydroxy-24-methyl-B-homo-7-oxa-5α-cholestan-6-one (9) were synthesized or purchased previously (Esposito, supra), and are shown in FIG. 7. All other chemicals and cell culture media were obtained from Sigma (Saint Louis, Mo.) or Invitrogen (Carlsbad, Calif.) unless specified otherwise.

Compound 5 in Table 6, (22S,23S)-6-aza-homobrassinolide, showed highest cytotoxicity with an IC₅₀ of 12.5 mM.

Two other synthetic brassinosteroids with fluorinated substitutes in the A ring of the molecule showed weak toxicity at 30 mM, the highest concentration tested. There was no apparent correlation between a compound's ability to induce cell proliferation and stimulation of cell migration (Table 6).

### Example 22

### Effect of Brassinosteroids on Scratch Wound Closure

To further examine effects of brassinosteroid treatment on scratch wound closure, 3T3 fibroblasts were examined after wounding and treatment with brassinosteroids.

### Scratch Wound Closure In Vitro

3T3 Swiss fibroblast were seeded into 24-well tissue culture at a concentration of 3×10⁵ cells/ml and cultured to nearly confluent cell monolayers. Then, a linear wound was generated in the monolayer with a sterile 100 μl plastic pipette tip. Any cellular debris was removed by washing with PBS. DMEM medium with vehicle (0.1% ethanol), FBS (1%, positive control), or various concentrations of brassinosteroids were added to a set of 3 wells per dose and incubated for 12 h at 37° C. with 5% CO₂. Cells were visualized in 10% methylene blue for 5 minutes. Three representative images from each well of the scratched areas under each condition were photographed to estimate the relative migration of cells at 0 and 12 h post-treatment. Data were analyzed using ImageJ software available online from the National Institutes of Health (rsbweb.nih.gov/ij/) by calculating the percentage of scratch closure at each dose point relative to control.

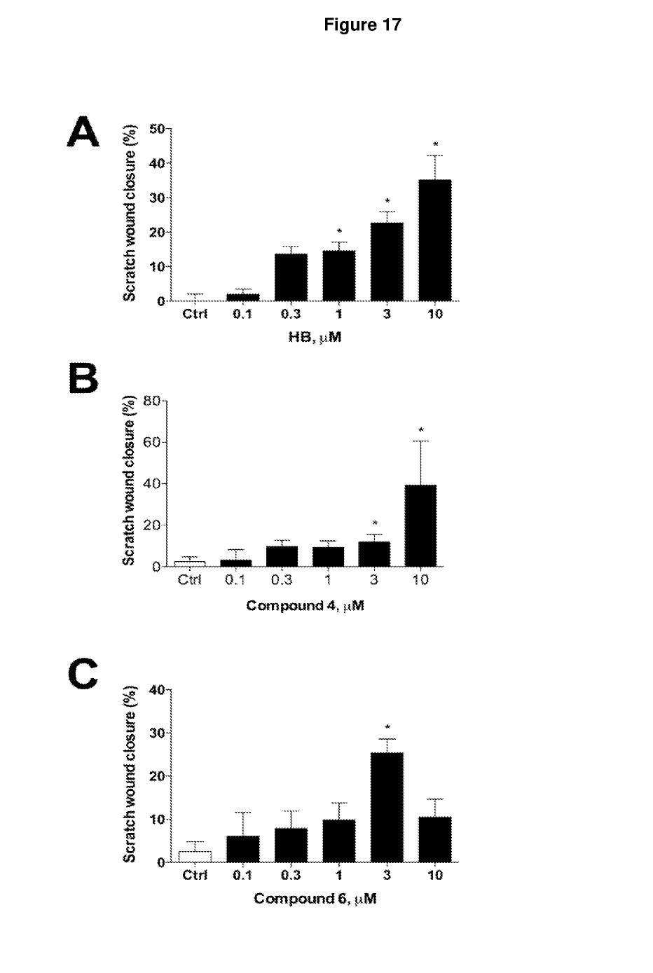

Microscopic observation of 3T3 fibroblasts demonstrated that HB promotes cell migration into a scratch wound zone with a maximum efficacy of 30±4.2% at 5 μM after 12 h of incubation. Such treatment with HB compares favorably to 1% FBS used as a positive control (41.5±6.5%), as well as reference activity of PDGF treatment reported previously (Schmidt et al., J. Ethnopharmacol. 122: 523-532, 2009). Several HB analogues showed similar or decreased scratch wound closure activity in this assay, with no specific reference to structural modifications (Table 6). The cytotoxic compound 5 ((22S,23S)-6-aza-homobrassinolide) showed no effect on fibroblast migration, as expected. A dose dependent migration activity was evaluated for all active compounds that significantly accelerated wound closure at concentrations of 0.1-10 μM. Compound 4 ((22S,23S)-3α-fluoro-homocastasterone) turned out to possess high activity, similar to HB, while compound 6 ((22S,23S)-7-aza-homobrassinolide) showed highest activity at 3 μM, possibly due to weak cytotoxicity associated with the higher doses of this treatment (FIG. 17A-C).

TABLE 6
Effect of HB (1) and its analogues (2-9) on cell viability, proliferation,
and scratch wound closure in 3T3 mouse fibroblasts.

| | | Scratch | Cell | |
|---|---|---|---|---|
| | | wound | proliferation | |
| | | closure at | at 5 μM, | Cell |
| | | 5 μM, % of | % of | cytotoxicity, |
| ID | Common name | control | control | IC₅₀ (μM) |
| 1 | (22S,23S)- | 30 ± 4.2* | 37.7 ± 3.4* | >30 |
| | homobrassinolide | | | |
| | (HB) | | | |
| 2 | (22S,23S)- | 32.9 ± 4.1* | Nt | >30 |
| | homocastasterone | | | |
| 3 | (22S,23S)-3α-fluoro- | 15.3 ± 2.6 | Nt | ~30 |
| | homobrasinolide | | | |
| 4 | (22S,23S)-3α-fluoro- | 27.7 ± 4.0* | Nt | ~30 |
| | homocastasterone | | | |
| 5 | (22S,23S)-6-aza- | 13.0 ± 0.7 | Nt | 12.5 |
| | homobrassinolide | | | |
| 6 | (22S,23S)-7-aza- | 30.5 ± 4.2* | Nt | >30 |
| | homobrassinolide | | | |
| 7 | (22R,23R)- | 16.5 ± 0.5 | 29.1 ± 3.4* | >30 |
| | homobrassinolide | | | |
| 8 | (22S,23S)- | 13.2 ± 1.5 | 31.9 ± 5.3* | >30 |
| | epibrassinolide | | | |
| 9 | (22R,23R)- | 8.1 ± 1.6 | 31.0 ± 2.6* | >30 |
| | epibrassinolide | | | |
| Ref | FBS, 1% | 41.5 ± 6.5* | 39.3 ± 5.7* | — |
| Ref | PDGF, 2 nM | 64.8 ± 1.7* | — | — |

Results are expressed as the mean ± SEM of determinations performed in triplicate (*P < 0.05 when compared with control by one-way ANOVA and Dunnett's post-test).
FBS and platelet-derived growth factor (PDGF) are shown as reference treatments.
Nt = not tested.

While HB showed no cytotoxicity in vitro when tested up to a concentration of 30 μM, several brassinosteroid analogues containing either a 6-aza group in the B ring of the molecule, or fluorinated substitutes in the A ring, showed weak toxicity at the highest concentrations tested (Table 6). All four brassinosteroids tested in this study for their ability to induce cell proliferation at 5 μM, showed moderate biological activity that had no correlation to structural changes in either the A or B ring of the molecule (Table 6). There was also no correlation between a compound's ability to induce cell proliferation and its ability to stimulate cell migration, as both R,R- and S,S-24-epibrassinolides promoted cell proliferation but not migration, while HB treatment resulted in significant increases in both parameters. There was a direct correlation, however, between a compound's ability to promote cell migration (FIG. 17) and its ability to induce phosphorylation of Akt (Esposito, supra), a key enzyme in signal transduction pathways involved in cell survival, cell-cycle progression, and migration.

The disclosure has been described in terms of particular embodiments found or proposed to comprise specific modes for the practice of the disclosure. Various modifications and variations of the described disclosure will be apparent to those skilled in the art without departing from the scope and spirit of the disclosure. Although the disclosure has been described in connection with specific embodiments, it should be understood that the subject matter of the disclosure as claimed should not be unduly limited to such specific embodiments. Indeed, various modifications of the described modes for carrying out the disclosure would be apparent to those skilled in the relevant fields are intended to be within the scope of the following claims.

1. A method for increasing a whole-body anabolic effect in a subject comprising the step of administering to the subject a therapeutically effective amount of a composition comprising a brassinosteroid compound.

2. The method of claim 1 wherein the brassinosteroid compound is a compound of formula I or a derivative thereof: 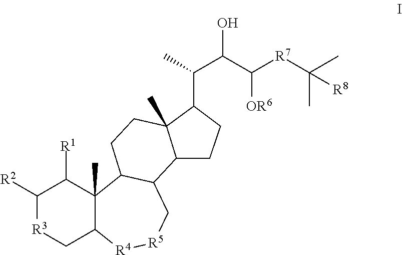
wherein:
R¹ and R² are each independently selected from the group consisting of H and OH;
R³ is selected from the group consisting of C(H)OH, C(H)F, C═O, and C(H)OR⁹;
or R² and R³ together with the carbon atom to which they are bonded form a 3-membered epoxide ring;
R⁴ is selected from the group consisting of CH₂, C═O, C(H)OH, and NH;
R⁵ is selected from the group consisting of a bond, O, NH, and C═O;
R⁶ is selected from the group consisting of H and 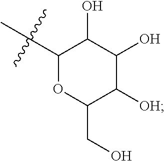 [SMILES: *C1OC(CO)C(O)C(O)C1O]
R⁷ is selected from the group consisting of CH₂, C(H)CH₃, C(H)CH₂CH₃, C═CH₂, and C═C(H)CH₃;
R⁸ is selected from the group consisting of H and CH₃;
R⁹ is selected from the group consisting of C(═O)(CH₂)ₙCH₃ and 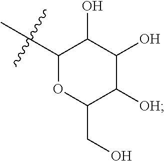 [SMILES: *C1OC(CO)C(O)C(O)C1O]
and
n is selected from the group consisting of 0, 1, 2, 3, 4, 5, 6, 7, 8, 9, 10, 11, 12, 13, 14, 15, 16, 17, and 18.

3. The method of claim 1 wherein the brassinosteroid compound is selected from the group consisting of: (22S,23S)-homobrassinolide (HB), (22S,23S)-homocastasterone, (22S,23S)-3α-fluoro-homobrasinolide, (22S,23S)-3α-fluoro-homocastasterone, (22S,23S)-6-aza-homobrassinolide, (22S,23S)-7-aza-homobrassinolide, (22R,23R)-homobrassinolide, (22S,23S)-epibrassinolide, and (22R,23R)-epibrassinolide.

4. The method of claim 1 wherein the whole-body anabolic effect comprises minimal or no androgenic side effect.

5. The method of claim 1 wherein the whole-body anabolic effect is an anabolically favorable state for muscle or skin.

6. The method of claim 5 wherein the anabolically favorable state for muscle is measured by increased protein synthesis, increased protein accumulation, or decreased protein degradation in muscle cells.

7. The method of claim 5 wherein the anabolically favorable state for muscle is measured by increased skeletal muscle mass.

8. The method of claim 7 wherein the increased skeletal muscle mass is measured by an increased total number of muscle fibers or by an increased cross-sectional area of muscle fibers.

9. The method of claim 8 wherein the increased number of muscle fibers is measured by increased type I and/or type II muscle fibers.

10. The method of claim 5 wherein the anabolically favorable state for muscle is measured by increased lean body mass, increased body weight gain, and/or decreased fat mass.

11. The method of claim 5 wherein the anabolically favorable state for muscle is measured by increased physical performance, increased physical strength, and/or increased physical fitness.

12. The method of claim 11 wherein the increased physical strength is measured by increased grip strength.

13. The method of claim 5 wherein the anabolically favorable state for muscle is measured by increased phosphorylation of alpha serine/threonine-protein kinase (AKT).

14. The method of claim 5 wherein the anabolically favorable state for skin is measured by increased protein synthesis, increased protein accumulation, decreased protein degradation in skin cells, or decreased wound healing time.

15. The method of claim 14 wherein the increased protein synthesis is measured by increased collagen production.

16. The method of claim 14 wherein the increased protein accumulation or the decreased protein degradation is measured by increased collagen.

17. The method of claim 14 wherein the increased protein synthesis is measured by increased elastin production.

18. The method of claim 14 wherein the increased protein accumulation or the decreased protein degradation is measured by increased elastin.

19. The method of claim 14 wherein the decreased wound healing time is measured by time at which 50% of a cutaneous wound is closed.

20. The method of claim 14 wherein the decreased wound healing time is measured by percent of original wound size.

21. The method of claim 14 wherein the decreased wound healing time results from decreased inflammation.

22. The method of claim 21, wherein the decreased inflammation is measured by decreased expression of TGF-β messenger RNA, decreased expression of TNF-α messenger RNA, or decreased expression of ICAM-1 messenger RNA.

23. The method of claim 5 wherein the anabolically favorable state for skin is demonstrated by skin that has increased elasticity, increased smoothness, reduced wrinkles, and/or improved color attributable to healthy infusion of blood.

24. The method of claim 1 wherein the brassinosteroid compound is administered at least weekly to the subject at a dosage from about 0.1 mg/kg to about 1000 mg/kg.

25. The method of claim 1 wherein the brassinosteroid compound is administered daily to the subject at a dosage from about 0.1 mg/kg to about 1000 mg/kg.

26. The method of claim 1 wherein the brassinosteroid compound is administered twice daily at a dosage from about 0.1 mg/kg to about 1000 mg/kg.

27. The method of claim 1 wherein the brassinosteroid compound is administered over a period of time from days to weeks or from days to months.

28. The method of claim 1 wherein the compound is administered topically, parenterally, or enterally.

29. The method of claim 28 wherein the topical administration is to the skin for cosmetic use.

30. The method of claim 1 wherein the subject is a mammal.

31. The method of claim 30 wherein the mammal is a human.


SEQUENCE LISTING (6 sequences)

SEQ ID NO: 1 - DNA, 24 bp, Artificial Sequence
  Synthetic primer
acgttgctatccaggctgtgctat

SEQ ID NO: 2 - DNA, 26 bp, Artificial Sequence
  Synthetic primer
ctcggtgaggatcttcatgaggtagt

SEQ ID NO: 3 - DNA, 20 bp, Artificial Sequence
  Synthetic primer
aagcagcagcaaagttcggt

SEQ ID NO: 4 - DNA, 23 bp, Artificial Sequence
  Synthetic primer
actaagcctgcagcagctccata

SEQ ID NO: 5 - DNA, 20 bp, Artificial Sequence
  Synthetic primer
gggaaatcgtgcgtgacatt

SEQ ID NO: 6 - DNA, 17 bp, Artificial Sequence
  Synthetic primer
gcggcagtggccatctc